(19) United States
(12) Patent Application Publication
(10) Pub. No.: US 2025/0195652 A1
(43) Pub. Date: Jun. 19, 2025

(54) Method for Engineering Innate-Like Lymphocytes

(71) Applicant: UCL BUSINESS LTD, London (GB)

(72) Inventors: Jonathan Fisher, London (GB); Marta Barisa, London (GB); Daniel Fowler, London (GB); John Counsell, London (GB)

(21) Appl. No.: 18/849,615

(22) PCT Filed: Mar. 24, 2023
(86) PCT No.: PCT/GB2023/050752

§ 371 Date: Sep. 23, 2024

(30) Foreign Application Priority Data
Mar. 25, 2022 (GB) 2204272.5

(51) Int. Cl. A61K 40/11; A61K 40/15; C12N 5/0783

(52) U.S. Cl. CPC A61K 40/11; A61K 40/15; C12N 5/0636; C12N 5/0646; C12N 2510/00; C12N 2740/15043

(57) ABSTRACT

The present invention relates to methods for introducing nucleic acid into innate-like lymphocytes, including gamma delta T cells, with a lentiviral vector; populations or compositions of genetically engineered innate-like lymphocytes; and methods for treating or preventing disease.

### FIELD OF THE INVENTION

The present invention relates to methods for introducing nucleic acid into innate-like lymphocytes, including gamma delta T cells, with a lentiviral vector; populations or compositions of genetically engineered innate-like lymphocytes; and methods for treating or preventing disease.

### BACKGROUND

Adoptive cell therapy or transfer (ACT) is a form of immunotherapy involving the isolation of T cells from a patient or donor, genetically modifying and/or expanding said cells, and subsequently reinfusing them back to the patient. For example, T cells may be engineered to express a tumour antigen specific T cell receptor (TCR) or chimeric antigen receptor (CAR), in order to redirect the immune response toward tumour cells.

The majority of cellular engineering approaches to date have been applied to as T cells, which are easy to expand and purify from peripheral blood. However, such approaches have limitations.

For example, the use of TCR requires the identification of a HLA-matched TCR against a processed antigen presented by tumour cells, and is susceptible to tumour immune-evasion strategies such as downregulation of MHC. Furthermore, there is a risk of graft versus host disease (GvHD) when using donor (allogeneic) cells. While the use of CAR may remove the need for HLA-matching and antigen presentation on tumour MHC, there remains an opportunity for immune-evasion through antigen loss.

The use of innate or innate-like immune lymphocytes, such as γδ T and natural killer (NK) cells, may offer several potential advantages. Firstly, tumour recognition and killing is not dependent on the expression of a single antigen, as innate immune cells can recognise a broad spectrum of antigens through their innate receptors. This property reduces the chance of immune escape by single antigen loss and provides an opportunity for targeting tumours lacking well-defined neo-antigens. Secondly, innate immune cells recognise their target cells in an MHC-independent manner, thus reducing the risk of alloreactivity and GvHD, and enabling the development of “off-the-shelf” allogeneic therapies.

Combining the properties of innate-like immune cells with engineering strategies to enhance cytotoxicity and/or redirect the cells toward specific targets is therefore a promising strategy for ACT.

The clinical and commercial gold standard for lentiviral-based cell therapy comprises VSVg-pseudotyped lentiviruses. Use of non-VSVg-pseudotyped lentiviruses clinically and translationally is so rare that few viral manufacturers (in the form of contract research organisations (CRO's) or contract development and manufacturing organizations (CDMO's)) offer such products in their repertoire. This is largely due to the wide and successful use of VSVg-pseudotyped lentiviruses for canonical, autologous as T cell clinical use.

While capable of efficient delivery of transgenes to activated canonical αβ T cells, VSVg-pseudotyped lentiviruses are poor at transducing innate-like lymphocytes, such as γδ T cells and NK cells. This is in large part due to the low expression of the VSVg entry receptor (low density lipoprotein or ‘LDL’ receptor) on innate lymphocytes. A recent report on optimised manufacture of γδ T cells for immunotherapeutic use recommended a multiplicity of infection (MOI) of 80 to transduce expanding γδ T cells with VSVg-pseudotyped lentivirus; moreso, even with this MOI, the group reported an average transduction efficiency of only 20% (Sutton et al., 2016, Cytotherapy, 18(7): 881-892). A recent report aimed at optimising NK cell transduction with VSVg-pseudotyped lentiviruses reported 11.8% transduction efficiency of activated cells with a viral MOI of 10, and went on to compare various transduction enhancers to boost this efficiency, including commonly used boosters such as vectofusin, dextran and PGE2. They concluded that of all the boosters screened, statins led to the greatest enhancement of transduction efficiency, yielding a 33.3% transduction efficiency with rosuvastatin (Gong et al., 2020, Mol Ther Methods Clin Dev, 17: 634-646). In both cases, the use of high viral MOI and/or transduction boosters came with an expected cost to the quality and numbers of the cell product. Significantly, statins were found to be inhibitory to NK cell cytotoxicity. Furthermore, the requirement for large batches of virus (for high MOI transductions) and access to transduction boosters entails high costs and strains on cell therapy manufacture supply chains.

This highlights the unmet need for efficient and cost-effective innate-like lymphocyte lentiviral transduction methods that are not injurious to cell quality or numbers.

### SUMMARY OF THE INVENTION

The invention provides methods for the efficient introduction of genetic material into innate-like lymphocytes with a lentiviral vector that has been pseudotyped with a RD144-HIV chimeric envelope protein (also known as ‘RD-Pro’). The inventors have found that chimeric RD144-HIV pseudotyped lentivirus is surprisingly effective at delivering transgenes to stimulated innate-like lymphocytes at low MOI and with unexpectedly favourable cell viability and proliferation. As mentioned earlier, VSVg-based transduction protocols can require MOIs of up to 80. In contrast, chimeric RD144-HIV pseudotyped lentiviral vectors can achieve transduction efficiencies of approximately 70% at MOIs as low as 1.25, thus requiring substantially lower viral titres (up to a 60-fold lower viral requirement). Furthermore, transduction with chimeric RD144-HIV pseudotyped virus presents with a fraction of the toxicity, resulting in higher levels of cell product that is healthier and more effective. Accordingly, the methods of the present invention enable a more cost-effective product manufacture process that further yields cells of better quality than standard lentiviral protocols.

The invention provides a method for introducing nucleic acid into innate-like lymphocytes with a lentiviral vector, wherein the lentiviral vector comprises a RD114-HIV chimeric envelope protein.

Some have previously described the use of alternative RD114-derived lentiviral pseudotypes for γδ T cell transduction, namely RD114TR-pseudotyped lentivirus (WO 2019/104269). Interestingly (and in contrast to our findings with RD114-HIV chimeric envelope protein or ‘RD-Pro’), VSVg-pseudotyped lentivirus led to greater transgene expression at day 5 post-transduction compared to RD114TR-pseudotyped lentivirus. This alludes to the fact that not all RD114-derived lentiviral envelopes confer similar enhancement to innate lymphocyte transduction as the RD-Pro envelope.

The innate-like lymphocytes may be γδ T cells, natural killer (NK) cells, natural killer T (NKT) cells, invariant natural killer T (iNKT) cells, mucosal-associated invariant T (MAIT) cells, lymphoid tissue inducer (LTi) cells, intra-epithelial lymphocytes (IELs), innate lymphoid cells (ILC), such as group 1 innate lymphoid cells, group 2 innate lymphoid cells, group 3 innate lymphoid cells, 1 cells, marginal zone B cells, and/or cells expressing CD8αα.

The innate-like lymphocytes may be γδ T cells, natural killer (NK) cells, natural killer T (NKT) cells, mucosal-associated invariant T (MAIT) cells, lymphoid tissue inducer (LTi) cells, intra-epithelial lymphocytes (IELs), innate lymphoid cells (ILC), including group 1 innate lymphoid cells, group 2 innate lymphoid cells, group 3 innate lymphoid cells, 1 cells, marginal zone B cells, and/or cells expressing CD8αα.

The innate-like lymphocytes may be γδ T cells, natural killer (NK) cells, invariant natural killer T (iNKT) cells, mucosal-associated invariant T (MAIT) cells, lymphoid tissue inducer (LTi) cells, intra-epithelial lymphocytes (IELs), innate lymphoid cells (ILC), such as group 1 innate lymphoid cells, group 2 innate lymphoid cells, group 3 innate lymphoid cells, 1 cells, marginal zone B cells, and/or cells expressing CD8αα.

The innate-like lymphocytes may be γδ T cells, natural killer (NK) cells, mucosal-associated invariant T (MAIT) cells, lymphoid tissue inducer (LTi) cells, intra-epithelial lymphocytes (IELs), innate lymphoid cells (ILC), including group 1 innate lymphoid cells, group 2 innate lymphoid cells, group 3 innate lymphoid cells, 1 cells, marginal zone B cells, and/or cells expressing CD8αα. In some embodiments, the innate-like lymphocytes are γδ T cells, such as Vδ2+ T cells.

In some embodiments, the innate-like lymphocytes are γδ T cells, such as Vδ1+ T cells.

In some embodiments, the innate-like lymphocytes are γδ T cells, such as non-Vδ1+/Vδ2+ T cells.

In some embodiments, the innate-like lymphocytes are NK cells.

In some embodiments, the chimeric envelope protein comprises a RD114 envelope protein, wherein the R peptide cleavage sequence is replaced with a HIV-1 matrix/capsid cleavage sequence.

The RD114-HIV chimeric envelope protein may have the sequence of SEQ ID NO: 4.

The invention provides a method for introducing nucleic acid into innate-like lymphocytes with a lentiviral vector at low multiplicities of infection (MOI).

In some embodiments, the nucleic acid is introduced in to the innate-like lymphocytes by the lentiviral vector as described herein at an MOI of around 50, around 25, around 10, around 5, around 2.5 or around 1.25.

In some embodiments, the nucleic acid is introduced in to the innate-like lymphocytes by the lentiviral vector as described herein, in the absence of transduction boosters.

The invention provides a method for introducing a gene of interest in to innate-like lymphocytes. The gene of interest may comprise any therapeutically relevant nucleotide sequence.

For example, the gene may encode an immunologically or metabolically active, naturally occurring or synthetic protein or chimeric molecule, of human or non-human origin. The encoded protein may be expressed intracellularly, at the cell membrane or secreted from the cell.

In some embodiments, the lentiviral vector described herein comprises a gene encoding a chimeric antigen receptor (CAR), chimeric co-stimulatory receptor, dual-CAR, tri-CAR, tether-CAR, non-signalling CAR, truncated CAR, T cell receptor (TCR), TCR co-expressed with CAR, scFv-Fc fusion protein (SFP), antibody, DARPIN, nanobody, tribody, duabody, bi-specific T-cell engager (BiTE), transcription factor, intracellular signalling molecule and/or mediator, cytokine, chemokine, integrin, lectin, adhesion molecule, cell surface receptor, cell surface ligand, glucose transporter, ion transporter, membrane-proximal and/or membrane-distal intracellular enzyme, such as a phosphatase, tyrosine kinase, serine threonine kinase, protease, matrix metalloproteinase, and/or degron.

The invention provides a method for introducing nucleic acid into innate-like lymphocytes, comprising the steps of:
(i) obtaining a population of innate-like lymphocytes; and
(ii) incubating the innate-like lymphocytes with a lentiviral vector;
   wherein the lentiviral vector comprises a RD114-HIV chimeric envelope protein.

The method may be carried out in the absence of transduction enhancer.

The population of innate-like lymphocytes may be isolated from a subject, for example from a blood or tissue-derived product.

The methods of the invention may further comprise a step of stimulating, such as activating and/or expanding, the innate-like lymphocytes prior to, and/or during incubation, with lentiviral vector.

The innate-like lymphocytes may be activated and/or expanded in the presence of zoledronic acid, interleukin 2 (IL-2), interleukin 15 (IL-15), and/or anti-CD3 antibody.

The invention provides a population of genetically engineered innate-like lymphocytes obtained or obtainable by the methods of the present invention.

The invention provides a composition, such as a pharmaceutical composition, comprising an innate-like lymphocyte population according to the present invention.

The innate-like lymphocytes may be genetically modified to enhance their anti-tumour properties (such as cytotoxicity and cytokine production), proliferation, persistence, metabolic fitness (for example resistance to hypoxia or glucose starvation), ability to distinguish between healthy and malignant tissue, anti-inflammatory properties, and/or resistance to chemotherapeutic drugs.

For example, the innate-like lymphocytes may be genetically modified to express a chimeric antigen receptor (CAR), chimeric co-stimulatory receptor, dual-CAR, tri-CAR, tether-CAR, non-signalling CAR, truncated CAR, T cell receptor (TCR), TCR co-expressed with CAR, scFv-Fc fusion protein (SFP), antibody, DARPIN, nanobody, tribody, duabody, bi-specific T-cell engager (BiTE), transcription factor, intracellular signalling molecule and/or mediator, cytokine, chemokine, integrin, lectin, adhesion molecule, cell surface receptor, cell surface ligand, glucose transporter, ion transporter, membrane-proximal and/or membrane-distal intracellular enzyme, such as a phosphatase, tyrosine kinase, serine threonine kinase, protease, matrix metalloproteinase, and/or degron. The invention provides a method of treating or preventing a disease in a subject, comprising administering the innate-like lymphocyte population or composition according to the present invention to a subject.

The invention provides an innate-like lymphocyte population or composition according to the present invention, for use in treating or preventing a disease in a subject.

The invention provides an innate-like lymphocyte population or composition according to the present invention, for use in immunotherapy.

The invention provides use of an innate-like lymphocyte population or composition according to the present invention, for the preparation of a medicament for treatment or prevention of a disease in a subject.

The innate-like lymphocytes may be allogeneic.

The disease may be a cancer; an autoimmune disease or an immune pathology; an infectious disease, such as a bacterial, fungal or viral infection; associated with organ transplant, such as GvHD; or a wound, ulcer, or abscess.

These and other aspects and embodiments of the invention will be described in further detail below.

### BRIEF DESCRIPTION OF THE FIGURES

FIG. 1—(A) Transduction efficiency of CD3+/Vδ2+ and CD3+/Vδ2− cells at day 7 post-transduction, comparing transduction with RD114-HIV chimeric envelope (also known as ‘RD-Pro’) pseudotyped lentivirus and VSVg pseudotyped lentivirus, both at MOI 15, using eGFP expression as a reporter of transduction. (B) Live Vδ2+ cells per ml at day 7 post-transduction in the same conditions as in (A). (C) Live GFP expressing V62+ cells per ml at day 7 post-transduction, in the same conditions as in (A) and (B).

FIG. 2—(A) Flow cytometry analysis of CD3 and V62 expression at D4, D8 and D11 post-transduction with RD114-HIV chimeric envelope (‘RD-Pro’) pseudotyped lentivirus. (B) GFP expression in CD3+V62+ cells. (C) % V62+ cells expressing GFP at different MOIs on days 4, 8 and 11 post-transduction.

FIG. 3—(A) Example of a method for expansion and transduction of a mixed population of γδ T cells. (B) Transduction efficiency of Vδ1+, Vδ2+ and Vδ1−/Vδ2− cells 12 days after transduction with a RD114-HIV chimeric envelope (‘RD-Pro’) pseudotyped lentivirus at MOI 4, containing a bicistronic expression cassette including an eGFP reporter.

FIG. 4—(A) Transduction efficiency of Vδ2+ cells with RD114-HIV chimeric envelope (‘RD-Pro’) pseudotyped lentivirus at a range of MOIs. One lentivirus (14G2a-SFP) encodes a bicistronic construct comprising eGFP and a secreted GD2-binding opsonin, the other (104) encodes a bicistronic construct comprising eGFP and luciferase. (B) Vδ2+ transduction efficiency (% GFP expressing cells) following transduction with the 14G2a-SFP lentivirus as in (A). (C) Percentages of live cells which are Vδ2+/GFP+ cells following transduction as in (A). (D) Flow cytometry analysis of CD3 and Vδ2 expression of live cells transduced with 14G2a-SFP lentivirus as in (A). (E) Detection of the anti-GD2 opsonin in supernatant of γδ T cells transduced with 14G2a-SFP encoding lentivirus as in (A), demonstrating the relationship between viral MOI and the amount of opsonin detected by binding to GD2+ target cells.

FIG. 5—(A) Similar to the γδ T cells above, NK cells (CD3-CD56+) in expansion were transduced with an RD114-HIV chimeric envelope protein (‘RD-Pro’)-pseudotyped lentivirus at MOI 4 and in the absence of transduction boosters on day 2 after stimulation. NK cells were transduced with a bicistronic GFP-secreted mitogen construct, and GFP expression was measured on day 14 of expansion. Representative data from 3 different donors is shown, with a mean transduction efficiency of 55%. (B) Efficient NK cell transduction could also be achieved using a different activation method. Representative transduction efficiency data is shown from one donor of either non-transduced cells or cells transduced with two different RD-Pro-pseudotyped bicistronic GFP-mitogen constructs. As above, NK cells were transduced on day 2 after culture initiation in the absence of transduction boosters and with an MOI of 4.

FIG. 6—(A and B) Example expansion protocol for γδ T cells. (C) Transduction efficiency (GFP transgene expression) of Vδ1+, Vδ2+ and Vδ1−/Vδ2− cells from different expansion protocols 12 days after transduction with RD-Pro-pseudotyped lentivirus at MOI 4.

FIG. 7—(A) NK cells (CD3-CD56+) in zoledronic acid-activated PBMC were transduced with RD-Pro- and VSVg-pseudotyped lentivirus at MOI 2. GFP expression was measured on day 14 of expansion. Representative data from 3 different donors is shown. (B) High transduction was maintained with large bicistronic and immunologically active tricistronic vectors at low MOI when NK cells were activated with proprietary, feeder-free and CD2/NKp46 GMP-compatible NK cell manufacturing kits from Miltenyi Biotec.

FIG. 8—(A) Transduction efficiency (GFP expression) at day 14 of NK-T cells in zoledronic acid-activated PBMC transduced with MOI 4 RD-Pro lentivirus. (B) High NK-T cell transduction was maintained with large bicistronic and immunologically active tricistronic vectors at low MOI when NK cells were activated with proprietary, feeder-free and CD2/NKp46 GMP-compatible NK cell manufacturing kits from Miltenyi Biotec.

### DETAILED DESCRIPTION OF THE INVENTION

### Innate-Like Lymphocytes

The invention relates to methods for introducing a nucleic acid into innate-like lymphocytes with a lentiviral vector comprising a RD114-HIV chimeric envelope protein.

The term “innate-like lymphocytes” as used herein refers to lymphocytes displaying innate-like immune properties. The antigen recognition and effector functions of innate immune cells is typically mediated via germline encoded receptors. In contrast, adaptive immune responses rely on gene rearrangements and somatic hypermutation in order to generate diverse antigen recognition. Innate-like lymphocytes are considered to bridge the gap between innate and adaptive immunity, for example, innate-like lymphocytes may express semi-invariant antigen receptors.

Examples of innate-like lymphocytes include γδ T cells, natural killer (NK) cells, natural killer T (NKT) cells, invariant natural killer T (iNKT) cells, mucosal-associated invariant T (MAIT) cells, lymphoid tissue inducer (LTi) cells, intra-epithelial lymphocytes (IELs), innate lymphoid cells, such as group 1 innate lymphoid cells, group 2 innate lymphoid cells, group 3 innate lymphoid cells, 1 cells, marginal zone B cells, and/or cells expressing CD8αα.

In some embodiments, the innate-like lymphocytes do not express an as T cell receptor.

In some embodiments, the innate-like lymphocytes are not canonical as T cells.

In some embodiments, the innate-like lymphocytes express a semi-invariant as T cell receptor.

γδ T cells represent a subset of T cells expressing the γδ TCR instead of the αβ TCR. Human γδ T cells normally comprise only 1-5% of circulating T lymphocytes but undergo rapid expansion in response to tumour, inflammation, and invading pathogens.

γδ T cells can be divided into two primary subsets—the tissue-bound Vδ1-positive cells and the peripheral circulating Vδ2-positive cells. The Vδ2 chain typically pairs with the Vγ9 chain, and Vγ9/Vδ2 T cells account for 50-95% of the peripheral T cells within the 1-5% T cells in circulation. Vδ1 γδ T cells represent the predominant T cell subset in solid tissues and the second most frequent subset in peripheral blood (1-3% of lymphocytes) next to Vγ9/Vδ2 T cells. Vδ1 T cells mainly reside in mucosal epithelial tissues, comprising approximately 40% of all intra-epithelial lymphocytes in the large intestine. Both Vδ1 and Vδ2 subsets have been shown to have anti-viral and anti-tumour activities. Unlike the conventional αβ TCR expressing cells, γδ TCR-expressing cells recognise their targets independent of the classical MHC I and II.

Similar to natural killer (NK) T cells, γδ T cells express NKG2D, which binds to the non-classical MHC molecules, i.e., MHC class I polypeptide-related sequence A (MICA) and MHC class I polypeptide-related sequence B (MICB), present on stressed cells and/or tumour cells. The γδ TCR recognises a variety of ligands, e.g., stress and/or tumour-related phosphoantigen. γδ T cells mediate direct cytolysis of their targets via multiple mechanisms, i.e., TRAIL, FasL, perforin and granzyme secretion. In addition, γδ T cells expressing CD16 potentiate antibody-dependent cell mediated cytotoxicity (ADCC).

In some embodiments, the innate-like lymphocytes according to the invention are γδ T cells.

In some embodiments, the innate-like lymphocytes according to the invention are Vδ2+ T cells, such as Vγ9/Vδ2 T cells.

In some embodiments, the innate-like lymphocytes are Vδ1+γδ T cells.

In some embodiments, the innate-like lymphocytes are non-Vδ1+Vδ2+γδ T cells, for example Vδ3+, Vδ4+, Vδ5+, Vδ6+, Vδ7+, or Vδ8+ T cells.

The δ-chain may pair with any suitable γ-chain, for example, Vγ2, Vγ3, Vγ4, Vγ5, Vγ8 or Vγ9.

Natural killer cells may be identified by the presence of CD56 and the absence of CD3. NK cells represent approximately 5-20% of all circulating lymphocytes in humans. NK cells are activated by cytokines including IL-12, IL-15, IL-18, IL-2, and CCL5. NK cells possess anti-tumour activity, mediated by receptors including NKG2D, NKp44, NKp46, NKp30, and DNAM, and can eliminate virus-infected cells via CD16-mediated ADCC.

Natural killer T (NKT) cells share properties of both T cells and natural killer cells. NKT cells may be identified by the presence of both CD3 and CD56, and an αβ TCR. They constitute approximately 1% of all peripheral blood T cells. Invariant natural killer T cells (also known as “type I” NKT cells) are a subset of NKT cells expressing an invariant TCRα chain and a limited number of non-invariant TCRβ chains (semi-invariant TCR). In humans, the highly conserved TCR is typically made of Va24-Ja18 segments paired with a Vδ11 chain. In contrast, type II NKT cells (also called diverse NKT or dNKT) use diverse TCRα and β chains.

Invariant natural killer T cells (iNKT) or type I NKT cells are a subset of T cells which are so named because they express cell surface markers associated with NK cells, but also possess an invariant or semi-invariant as T cell receptor (TCR). They represent approximately 0.1% of human T cells in peripheral blood. In contrast to classical T cells, iNKT cells recognize lipids and glycolipids presented by CD1d, a nonpolymorphic MHC protein expressed on intestinal epithelial cells. iNKT cells mediate their effector function primarily through rapid cytokine release following activation, including both Th1 (IFN-γ and TNF-α), Th2 (IL-1, IL-4, and IL-13), and Th17 (IL-17, IL-22) cytokines.

In some embodiments, the innate-like lymphocytes according to the invention are natural killer (NK) cells.

In some embodiments, the innate-like lymphocytes according to the invention are natural killer T (NKT) cells.

In some embodiments, the innate-like lymphocytes according to the invention are invariant natural killer T (iNKT) cells.

Mucosal-associated invariant T (MAIT) cells are important in the defence against bacteria at mucosal surfaces. They express an invariant TCR a chain and variable but restricted TCR p chains. MAIT cells are primarily found in mucosal tissues, such as the liver, lung, mesenteric lymph nodes, and intestinal epithelium. In human peripheral blood, they constitute approximately 1-10% of total T lymphocytes. MAIT cells express CD161, interleukin-18 receptor, and chemokine receptors CCR5, CXCR6, and CCR6 on the cell surface; and can be activated via the TCR or by IL-12 and IL-18 in a TCR-independent manner. MAIT cells are capable of releasing IFN-γ, TNF-α, and IL-17 in response to stimulation, and possess granzyme b and perforin mediated cytotoxic activity against infected cells.

In some embodiments, the innate-like lymphocytes according to the invention are MAIT cells.

Innate lymphoid cells (ILCs) is a collective term for cells with lymphoid morphology that do not contain rearranged antigen receptors and which lack myeloid-specific phenotypic markers. They do not directly recognise antigens, but instead respond to changes in cytokine expression profiles as a result of infection. ILCs are primarily tissue resident cells, found in both lymphoid and non-lymphoid tissues, and rarely in the peripheral blood. They are particularly abundant at mucosal surfaces, playing a key role in mucosal immunity and homeostasis.

Based on differences in developmental pathways, phenotype, and signalling molecules, ILCs are broadly divided into three groups. Group 1 ILC (ILC1), which includes NK cells, are responsive to cytokines such as IL-12 and IL-18, and produce IFN-γ. Group 2 ILC (ILC2) respond to IL-25, IL-33, and TSLP, and produce Th2 cytokines such as IL-4, IL-5 and IL-13. Group 3 ILCs (ILC3) express RORyt, and respond to IL-23 and IL-1p via the production of IL-22 and IL-17.

Lymphoid tissue inducer (LTi) cells share many similar characteristics with ILC3, however are generally considered a separate lineage due to their unique developmental pathway. Like ILC3s, LTi cells are dependent on RORyt. They are involved in the formation of secondary lymph nodes and Peyer's patches by promoting lymphoid tissue development. Activated LTi cells mostly produce IL-17A, IL-17F, and IL-22.

In some embodiments, the innate-like lymphocytes according to the invention are innate lymphoid cells, for example, group 1 innate lymphoid cells, group 2 innate lymphoid cells, group 3 innate lymphoid cells, and/or LTi cells.

Intraepithelial lymphocytes (IEL) refers to lymphocytes found in the epithelial layer of mucosal linings, such as the gastrointestinal and reproductive tracts. IELs can be divided into different subpopulations based on expression of TCR and CD8. Approximately 10-15% of IELs express the γδ TCR.

In some embodiments, the innate-like lymphocytes according to the invention are intra-epithelial lymphocytes.

CD8 is the co-receptor for TCR. CD8 is a dimer, consisting of a pair of CD8 chains. The most common form of CD8 comprises CD8a and CD8P chains, however homodimers of the CD8a chain are also expressed on some cells. For example, NK cells and γδ T cells almost exclusively express the CD8αα form. Expression of CD8αα is also an important phenotypic marker of IELs.

In some embodiments, the innate-like lymphocytes according to the invention express CD8αα.

Innate-like lymphocytes also include B cells expressing semi-invariant B cell receptors, such as B1 cells or marginal zone B cells. The B1 cell population comprises two subsets based on expression of CD5: B1a cells (CD5+) and B1b cells (CD5−). B1 cells generally express germline-encoded, polyreactive IgM antibodies with limited V gene segment usage. Marginal zone B cells are found in the spleen where they surround the follicles and are thus frequently exposed to blood-borne antigens. Following exposure to antigens, marginal zone B cells can present antigen and promote T cell activation, or may differentiate into plasmablasts. Marginal zone B cells express surface IgM, complement receptors CD35 and CD21, and the lipid antigen-presenting molecule CD1d.

In some embodiments, the innate-like lymphocytes according to the invention are 1 cells or marginal zone B cells.

The invention further provides a population of genetically engineered innate-like lymphocytes obtained or obtainable by the methods described herein.

The invention provides a population of innate-like lymphocytes, wherein said innate-like lymphocytes have been genetically engineered with a lentiviral vector comprising a RD114-HIV chimeric envelope protein.

The innate-like lymphocyte population or composition according to the invention may comprise at least about 10³ cells, at least about 10⁴ cells, at least about 10⁵ cells, at least about 10⁶ cells, at least about 10⁷ cells, or at least about 10⁸ cells, or at least about 10⁹ cells, or at least about 10¹⁰ cells, or at least about 10¹¹ cells.

The innate-like lymphocyte population or composition according to the invention may comprise at least about 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 96, 97, 98, 99 or 100% innate-like lymphocytes that have been genetically engineered according to the method of the invention.

### Lentiviral Vectors

Lentivirus vectors are part of the larger group of retroviral vectors. In brief, lentiviruses can be divided into primate and non-primate groups. Examples of primate lentiviruses include but are not limited to human immunodeficiency virus (HIV), the causative agent of human acquired immunodeficiency syndrome (AIDS), and simian immunodeficiency virus (SIV). Examples of non-primate lentiviruses include the prototype “slow virus” visna/maedi virus (VMV), as well as the related caprine arthritis-encephalitis virus (CAEV), equine infectious anaemia virus (EIAV), and the more recently described feline immunodeficiency virus (FIV) and bovine immunodeficiency virus (BIV).

The lentivirus family differs from retroviruses in that lentiviruses have the capability to infect both dividing and non-dividing cells. In contrast, other retroviruses, such as MLV, are unable to infect non-dividing or slowly dividing cells such as those that make up, for example, muscle, brain, lung and liver tissue.

A lentiviral vector, as used herein, is a vector which comprises at least one component part derivable from a lentivirus. Preferably, that component part is involved in the biological mechanisms by which the vector infects or transduces target cells with a gene of interest.

The term encompasses, for example, envelope-coated lipid nano- and micro-particles, vesicles and virus-like particles.

In one embodiment, the lentiviral vector is derived from HIV-1, HIV-2, SIV, FIV, BIV, EIAV, CAEV or Visna lentivirus.

The basic structure of retrovirus and lentivirus genomes share many common features such as a 5′ LTR and a 3′ LTR. Between or within these are located a packaging signal to enable the genome to be packaged, a primer binding site, integration sites to enable integration into a host cell genome, and gag, pol and env genes encoding the packaging components—these are polypeptides required for the assembly of viral particles. Lentiviruses have additional features, such as rev and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration and transcription. LTRs also serve as enhancer-promoter sequences and can control the expression of the viral genes.

The LTRs themselves are identical sequences that can be divided into three elements: U3, R and U5. U3 is derived from the sequence unique to the 3′ end of the RNA. R is derived from a sequence repeated at both ends of the RNA. U5 is derived from the sequence unique to the 5′ end of the RNA. The sizes of the three elements can vary considerably among different retroviruses.

In a defective retroviral or lentiviral vector genome gag, pol and env may be absent or not functional.

In a typical lentiviral vector as described herein, at least part of one or more coding regions for proteins essential for virus replication may be removed from the vector. This makes the viral vector replication-defective. Portions of the viral genome may also be replaced by a gene of interest in order to generate a vector comprising a gene of interest which is capable of transducing a target cell and/or integrating its genome into the target cell genome.

Optionally, the viral vector used in the present invention has a minimal viral genome.

By “minimal viral genome” it is to be understood that the viral vector has been manipulated so as to remove the non-essential elements and to retain the essential elements in order to provide the required functionality to infect, transduce and deliver a gene of interest to a target host cell. Further details of this strategy can be found in WO 1998/017815.

The lentiviral vector as described herein has been pseudotyped. In this regard, pseudotyping can confer one or more advantages. For example, the env gene product of HIV based vectors would restrict these vectors to infecting only cells that express a protein called CD4. However, if the env gene in these vectors has been substituted with env sequences from other enveloped viruses, then they may have a broader infectious spectrum (Verma and Somia (1997) Nature 389(6648):239-242).

In the present invention, the Env protein is a modified Env protein such as a mutant or engineered Env protein. Modifications may be made or selected to introduce targeting ability or to reduce toxicity or for another purpose (Valsesia-Wittman et al. 1996 J Virol 70: 2056-64; Nilson et al. (1996) Gene Ther 3(4):280-286; and Fielding et al. (1998) Blood 91(5):1802-1809 and references cited therein).

### RD114-HIV Chimeric Envelope Protein

The lentiviral vector for use in the methods of the present invention comprises a RD114-HIV chimeric envelope protein. In other words, the lentiviral vector of the present invention has been pseudotyped with a RD114-HIV chimeric envelope protein.

The term “envelope protein” or “Env” as used herein refers to the env gene product. The envelope protein is a glycoprotein located on the virion surface that facilitates transduction by binding receptors on the target cell surface and driving subsequent fusion of the target cell and viral membranes.

The term “chimeric envelope protein” as used herein refers to an envelope protein comprising a portion of protein derived from another protein. The other protein may or may not be an envelope protein. A chimeric envelope protein may be created, for example, by the joining of two or more genes that originally coded for separate proteins, such as two env genes.

RD114 refers to the feline leukemia virus RD114.

The RD114 receptor, neutral amino acid transporter (RDR), is widely expressed on hematopoietic stem cells. RD114 is a non-cytotoxic envelope protein, thus allowing the development of stable lentiviral producer cell lines.

An example of a chimeric RD114 envelope protein is a RD114 envelope protein comprising a replacement of the cytoplasmic tail region with a tail region from another viral envelope protein, such as the tail region of the murine leukemia virus (also known as ‘RD114TR’), for example as described in (WO 2019/104269).

An RD114-HIV chimeric envelope protein refers to a RD114 envelope protein comprising a portion of protein derived from HIV. For example, the HIV Env cytoplasmic tail or furin cleavage site.

In some embodiments, the RD114-HIV chimeric envelope protein comprises a RD114 envelope protein, wherein the R peptide cleavage sequence within the cytoplasmic tail is replaced with a HIV-1 matrix/capsid cleavage sequence, such as described in Bell et al. Experimental Biology and Medicine 2010; 235. This RD114-HIV chimeric envelope protein may also be referred to as RD-Pro.

It is known that replacing the protease cleavage sequence of the RD114 envelope protein with the HIV matrix/capsid cleavage sequence enhances the cleavage interactions between HIV-1 protease and the RD-Pro envelope protein during virion formation. The subsequent increase in cleavage correlates with enhanced titre values for RD-Pro-pseudotyped virus versus unmodified RD114-pseudotyped virus.

The RD114 envelope protein may comprise or consist of the sequence of SEQ ID NO: 1.

The RD114 envelope protein may have at least 70%, at least 75%, at least 80%, at least 85%, at least 90%, at least 95%, identity to SEQ ID NO: 1.

(RD114 envelope protein sequence)
SEQ ID NO: 1
MKLPTGMVILCSLIIVRAGFDDPRKAIALVQKQHGKPCECSGGQV
SEAPPNSIQQVTCPGKTAYLMTNQKWKCRVTPKISPSGGELQNCP
CNTFQDSMHSSCYTEYRQCRRINKTYYTATLLKIRSGSLNEVQIL
QNPNQLLQSPCRGSINQPVCWSATAPIHISDGGGPLDTKRVWTVQ
KRLEQIHKAMTPELQYHPLALPKVRDDLSLDARTFDILNTTFRLL
QMSNFSLAQDCWLCLKLGTPTPLAIPTPSLTYSLADSLANASCQI
IPPLLVQPMQFSNSSCLSSPFINDTEQIDLGAVTFTNCTSVANVS
SPLCALNGSVFLCGNNMAYTYLPQNWTRLCVQASLLPDIDINPGD
EPVPIPAIDHYIHRPKRAVQFIPLLAGLGITAAFTTGATGLGVSV
TQYTKLSHQLISDVQVLSGTIQDLQDQVDSLAEVVLQNRRGLDLL
TAEQGGICLALQEKCCFYANKSGIVRNKIRTLQEELQKRRESLAT
NPLWTGLQGFLPYLLPLLGPLLTLLLILTIGPCVFSRLMAFINDR
LNVVHAMVLAQQYQALKAEEEAQD

The R peptide cleavage sequence may consist of, or comprise, the sequence VHAMVLAQ (SEQ ID NO: 2). The R peptide cleavage sequence may have up to 5, up to 4, up to 3, up to 2, up to 1 amino acid substitutions relative to SEQ ID NO: 2.

The HIV-1 matrix/capsid cleavage sequence may consist of, or comprise, the sequence SQNYPIVQ (SEQ ID NO: 3). The HIV-1 matrix/capsid cleavage sequence may have up to 5, up to 4, up to 3, up to 2, up to 1 amino acid substitutions relative to SEQ ID NO: 3.

In some embodiments, the RD114-HIV chimeric envelope protein comprises the sequence of SEQ ID NO: 4.

In some embodiments, the RD114-HIV chimeric envelope protein consists of the sequence of SEQ ID NO: 4.

In some embodiments, the RD114 envelope protein has at least 50%, at least 60%, at least 70%, at least 75%, at least 80%, at least 85%, at least 90%, at least 95%, at least 96%, at least 97%, at least 98%, at least 99% identity to SEQ ID NO: 4.

(RD-Pro envelope protein sequence)
SEQ ID NO: 4
MKLPTGMVILCSLIIVRAGFDDPRKAIALVQKQHGKPCECSGGQV
SEAPPNSIQQVTCPGKTAYLMTNQKWKCRVTPKNLTPSGGELQNC
PCNTFQDSMHSSCYTEYRQCRANNKTYYTATLLKIRSGSLNEVQI
LQNPNQLLQSPCRGSINQPVCWSATAPIHISDGGGPLDTKRVWTV
QKRLEQIHKAMHPELQYHPLALPKVRDDLSLDARTFDILNTTFRL
LQMSNFSLAQDCWLCLKLGTPTPLAIPTPSLTYSLADSLANASCQ
IIPPLLVQPMQFSNSSCLSSPFINDTEQIDLGAVTFTNCTSVANV
SSPLCALNGSVFLCGNNMAYTYLPQNWTGLCVQASLLPDIDIIPG
DEPVPIPAIDHYIHRPKRAVQFIPLLAGLGITAAFTTGATGLGVS
VTQYTKLSHQLISDVQVLSGTIQDLQDQVDSLAEVVLQNRRGLDL
LTAEQGGICLALQEKCCFYANKSGIVRNKIRTLQEELQKRRESLA
SNPLWTGLQGFLPYLLPLLGPLLTLLLILTIGPCVFSRLMAFIND
RLNVSQNYPIVQQYQALKAEEEAQD*

It will be recognised by one of ordinary skill in the art that some amino acid sequences described herein can be varied without significant effect of the structure or function of the protein. If such differences in sequence are contemplated, a skilled person would understand how to determine which domains are critical areas to retain function of the protein.

It is known that certain amino acid changes are likely to be permissive at a certain position of the protein. For example, most buried amino acid residues require nonpolar side chains, whereas few features of surface side chains are generally conserved.

Typically seen as conservative substitutions are the replacements, one for another, among the aliphatic amino acids Ala (A), Val (V), Leu (L), and Ile (I); interchange of the hydroxyl residues Ser (S) and Thr (T), exchange of the acidic residues Asp (D) and Glu (E), substitution between the amide residues Asn (N) and Gln (Q), exchange of the basic residues Lys (K) and Arg (R), and replacements between the aromatic residues Phe (F) and Tyr (Y).

### Identity

The term “identity” as used herein, refers to the proportion of amino acids or nucleotides (expressed in percent) of a polypeptide or nucleotide sequence which are identical to a reference sequence.

The degree of sequence identity between a query sequence and a reference sequence may be determined by 1) aligning the two sequences by any suitable alignment program using the default scoring matrix and default gap penalty, 2) identifying the number of exact matches, where an exact match is where the alignment program has identified an identical amino acid or nucleotide in the two aligned sequences on a given position in the alignment and 3) dividing the number of exact matches with the length of the reference sequence.

Suitable computer programs for carrying out such an alignment include, but are not limited to, Vector NTI (Invitrogen Corp.) and the ClustalV, ClustalW and ClustalW2 programs (Higgins DG & Sharp PM (1988), Higgins et al. (1992), Thompson et al. (1994), Larkin et al. (2007). A selection of different alignment tools is available from the ExPASy Proteomics server at www.expasy.org. Another example of software that can perform sequence alignment is BLAST (Basic Local Alignment Search Tool), which is available from the webpage of National Center for Biotechnology Information which can currently be found at http://www.ncbi.nlm.nih.gov/ and which was firstly described in Altschul et al. (1990) J. Mol. Biol. 215; 403-410.

### Method of Introducing Nucleic Acid

The invention provides a method for introducing nucleic acid into innate-like lymphocytes. The method comprises the steps of:
(iii) obtaining a population of innate-like lymphocytes; and
(iv) incubating the innate-like lymphocytes with a lentiviral vector as described herein.

In some embodiments, the method is an in vitro method.

The lentiviral vector may comprise the nucleic acid. Accordingly, the nucleic acid may be introduced into the innate-like lymphocytes by the lentiviral vector, for example by transduction or infection, or by endocytosis or membrane fusion of envelope-coated lipid nano- or micro-particles, vesicles or virus-like particles.

An enveloped lentiviral particle may further deliver to the target cell something other than a nucleic acid, for example, a peptide, carbohydrate or lipid of interest.

The population of innate-like lymphocytes may be isolated from a subject or from a sample from a subject. The sample may be a peripheral blood sample, a cord blood sample, a tumour, a stem cell precursor, a tumor biopsy, a tissue, or a lymph. In some embodiments, the sample is a blood sample. In some embodiments, the sample is a tissue sample, such as skin or gut.

Peripheral blood mononuclear cells can be collected from a subject, for example, with an apheresis machine, including the Ficoll-Paque™ PLUS (GE Healthcare) system, or another suitable device/system. Innate-like lymphocytes may be purified from the collected sample with, for example, flow cytometry techniques. Cord blood cells may be obtained from cord blood during the birth of a subject.

In some embodiments, the innate-like lymphocytes may be isolated from a blood product, such as leukapheresate, leukocyte reduction system chamber (buffy cone), or buffy coat.

The cells may be autologous cells.

The cells may be allogeneic cells (i.e. derived from a donor).

The innate-like lymphocytes may be stimulated, for example activated and/or expanded, prior to and/or during incubation with a lentiviral vector.

Thus, the invention further provides a method for introducing a nucleic acid into innate-like lymphocytes, comprising the steps of:
(i) obtaining a population of innate-like lymphocytes, such as isolating a population of innate-like lymphocytes from a subject; and
(ii) incubating the innate-like lymphocytes with a lentiviral vector as described herein;
   or
(i) obtaining a population of innate-like lymphocytes, such as isolating a population of innate-like lymphocytes from a subject;
(ii) activating and/or expanding said innate-like lymphocytes; and
(iii) incubating the innate-like lymphocytes with a lentiviral vector as described herein.

In some embodiments, the innate-like lymphocytes are activated and/or expanded by culturing the cells in the presence of a bisphosphonate and/or one or more cytokines.

The bisphosphonate may be zoledronate (zoledronic acid), pamidronate, alendronate, risedronate, ibandronate, incadronate, clodronate, etidronate, or neridronate, a salt thereof and/or a hydrate thereof, preferably the bisphosphonate is zoledronic acid.

Suitable cytokines include interleukin (IL-) 2, IL-15, IL-12, IL-7, IL-21, IL-18, IL-19, IL-33, IL-4, IL-9, and/or IL-23.

In some embodiments, the cells are stimulated with a bisphosphonate and IL-2.

In some embodiments, the cells are stimulated with zoledronic acid and IL-2.

In some embodiments, the cells are stimulated with zoledronic acid, IL-2 and IL-15.

The concentration of bisphosphonate, such as zoledronic acid, during activation and/or expansion of the cells may be from about 0.1 μM to about 500 μM, from about 0.1 μM to about 400 μM, from about 0.1 μM to about 300 μM, from about 0.1 μM to about 200 μM, from about 0.1 μM to about 100 μM, from about 0.5 μM to about 100 μM, from about 1 μM to about 500 μM, from about 1 μM to about 400 μM, from about 1 μM to about 300 μM, from about 1 μM to about 200 μM, from about 1 μM to about 100 μM, from about 1 μM to about 90 μM, from about 1 μM to about 80 μM, from about 1 μM to about 70 μM, from about 1 μM to about 60 μM, from about 1 μM to about 50 μM, from about 1 μM to about 40 μM, from about 1 μM to about 30 μM, from about 1 μM to about 20 μM, from about 1 μM to about 15 μM, from about 1 μM to about 10 μM, from about 1 μM to about 9 μM, from about 1 μM to about 8 μM, from about 1 μM to about 7 μM, from about 1 μM to about 6 μM, from about 1 μM to about 5 μM, from about 2 μM to about 5 μM, from about 3 μM to about 5 μM, from about 2 μM to about 5 μM, from about 2 μM to about 10 μM, from about 3 μM to about 8 μM, or from about 4 μM to about 6 μM.

In a preferred embodiment, the concentration of bisphosphonate is 5 μM.

In a preferred embodiment, the concentration of zoledronic acid is 5 μM.

The concentration of IL-2 during activation and/or expansion of the cells may be from about 10 IU/ml to about 1000 IU/ml, from about 10 IU/ml to about 500 IU/ml, from about 10 IU/ml to about 400 IU/ml, from about 10 IU/ml to about 300 IU/ml, from about 10 IU/ml to about 200 IU/ml, from about 10 IU/ml to about 150 IU/ml, from about 10 IU/ml to about 100 IU/ml, from about 20 IU/ml to about 100 IU/ml, from about 30 IU/ml to about 100 IU/ml, from about 40 IU/ml to about 100 IU/ml, from about 50 IU/ml to about 100 IU/ml, from about 75 IU/ml to about 125 IU/ml, from about 20 IU/ml to about 80 IU/ml, from about 25 IU/ml to about 100 IU/ml, or from about 50 IU/ml to about 150 IU/ml.

In a preferred embodiment, the concentration of IL-2 is 100 IU/ml.

The concentration of IL-15 during activation and/or expansion of the cells may be from about 10 ng/ml to about 1 μg/ml, from about 10 ng/ml to about 500 ng/ml, from about 10 ng/ml to about 400 ng/ml, from about 10 ng/ml to about 300 ng/ml, from about 10 ng/ml to about 200 ng/ml, from about 10 ng/ml to about 150 ng/ml, from about 10 ng/ml to about 100 ng/ml, from about 20 ng/ml to about 100 ng/ml, from about 30 ng/ml to about 100 ng/ml, from about 40 ng/ml to about 100 ng/ml, from about 50 ng/ml to about 100 ng/ml, from about 60 ng/ml to about 100 ng/ml, from about 20 ng/ml to about 80 ng/ml, from about 30 ng/ml to about 60 ng/ml, or from about 50 ng/ml to about 120 ng/ml.

In a preferred embodiment, the concentration of IL-15 is 70 ng/ml.

In some embodiments, the cells are stimulated with an anti-CD3 antibody, for example OKT3, 145-2C11, 17A2 or a suitable alternative. Preferably, the anti-CD3 antibody is OKT3.

The concentration of anti-CD3 antibody during activation and/or expansion of the cells may be from about 0.1 μg/ml to about 50 μg/ml, from about 0.1 μg/ml to about 25 μg/ml, from about 0.1 μg/ml to about 10 μg/ml, from about 0.1 μg/ml to about 5 μg/ml, from about 0.1 μg/ml to about 2.5 μg/ml, from about 0.5 μg/ml to about 2.5 μg/ml, from about 0.5 μg/ml to about 2 μg/ml.

In a preferred embodiment, the concentration of anti-CD3 antibody (e.g. OKT3) is 1 μg/ml.

In some embodiments, the cells are stimulated with an anti-CD3 antibody (for example, OKT3 or another) and IL-2.

In some embodiments, the cells are stimulated with an anti-CD3 antibody, a bisphosphonate and IL-2.

In some embodiments, the cells are stimulated with an anti-CD3 antibody, a bisphosphonate, IL-2 and IL-15. The seeding density of the isolated cells during activation and/or expansion may be from about 0.01×10⁶ cells/cm² to about 1×10⁷ cells/cm², from about 0.1×10⁶ cells/cm² to about 5×10⁶ cells/cm², from about 0.25×10⁶ cells/cm² to about 5×10⁶ cells/cm², from about 0.5×10⁶ cells/cm² to about 5×10⁶ cells/cm², from about 0.5×10⁶ cells/cm² to about 4×10⁶ cells/cm², from about 0.5×10⁶ cells/cm² to about 3×10⁶ cells/cm², from about 0.5×10⁶ cells/cm² to about 2.5×10⁶ cells/cm², from about 0.5×10⁶ cells/cm² to about 2×10⁶ cells/cm², from about 0.6×10⁶ cells/cm² to about 2×10⁶ cells/cm², from about 0.7×10⁶ cells/cm² to about 2×10⁶ cells/cm², from about 0.8×10⁶ cells/cm² to about 2×10⁶ cells/cm², from about 0.9×10⁶ cells/cm² to about 2×10⁶ cells/cm², from about 1×10⁶ cells/cm² to about 2×10⁶ cells/cm², or from about 1.5×10⁶ cells/cm² to about 2×10⁶ cells/cm².

In some embodiments, the cells are stimulated, activated and/or expanded with an anti-CD2 antibody. In some embodiments, the cells are stimulated, activated and/or expanded with an anti-CD335 (NKp46) antibody. In some embodiments, the cells are stimulated, activated and/or expanded with an anti-CD2 and anti-CD335 (NKp46) antibody.

In a preferred embodiment, seeding density of the isolated cells during activation and/or expansion is 1×10⁶ cells/cm².

The duration of activation and/or expansion of the cells may be from 1 day to about 20 days, 1 day to about 15 days, 1 day to about 12 days, from 1 day to about 10 days, from 2 days to about 20 days, from 2 days to about 15 days, from 2 days to about 12 days, from 2 days to about 10 days, from 3 days to about 15 days, from about 4 days to about 15 days, from about 5 days to about 15 days, or from about 10 days to about 15 days.

In a preferred embodiment, the duration of activation and/or expansion of the cells is 12 days.

In some embodiments, the expansion and/or activation step comprises co-stimulatory agents. These co-stimulatory agents can include ligands binding to receptors expressed on innate-like lymphocytes, such as NKG2D, CD161, CD70, JAML, DNAX accessory molecule-1 (DNAM-1), ICOS, CD27, CD137, CD30, HVEM, SLAM, CD122, DAP, and CD28.

Example of reagents that can be used to facilitate the activation and/or expansion of innate-like lymphocytes may include anti-CD3, anti-CD2, anti-CD27, anti-CD30, anti-CD70, or anti-OX40 antibodies, IL-2, IL-15, IL-1 2, IL-9, IL-33, IL-1 8, or IL-21, CD70 (CD27 ligand), phytohaemagglutinin (PHA), concavalin A (ConA), pokeweed (PWM), protein peanut agglutinin (PNA), soybean agglutinin (SBA), Lens culinaris agglutinin (LCA), Pisum sativum agglutinin (PSA), Helix pomatia agglutinin (HPA), Vicia graminea Lectin (VGA), or another suitable mitogen capable of stimulating innate-like lymphocyte proliferation.

In some embodiments, cells expressing αβ TCR are depleted or reduced from the population of innate-like lymphocytes. For example using magnetic beads coated with anti-αβ TCR antibodies.

The methods of the present invention enable genetic engineering (e.g. by transduction) at low MOI.

In some embodiments, the innate-like lymphocytes are transduced with a lentiviral vector at an MOI of about 1 to about 100, about 1 to about 50, about 1 to about 25, about 1 to about 20, about 1 to about 15, about 1 to about 10, about 1 to about 5, or about 2 to about 4. In some embodiments, the innate-like lymphocytes are transduced with a lentiviral vector at an MOI of about 0.5 to about 5. In some embodiments, the innate-like lymphocytes are transduced with a lentiviral vector at an MOI of about 0.5 to about 2.5. In some embodiments, the innate-like lymphocytes are transduced with a lentiviral vector at an MOI of about 1 to about 2.5.

In some embodiments, the innate-like lymphocytes are transduced with a lentiviral vector at an MOI of less than 5.

In some embodiments, the innate-like lymphocytes are transduced with a lentiviral vector at an MOI of less than 3.

In some embodiments, the innate-like lymphocytes are transduced with a lentiviral vector at an MOI of less than 2.5.

In some embodiments, the innate-like lymphocytes are transduced with a lentiviral vector at an MOI of less than 2.

In some embodiments, the innate-like lymphocytes are transduced with a lentiviral vector at an MOI of less than 1.5.

In some embodiments, the innate-like lymphocytes are transduced with a lentiviral vector at an MOI of less than 1.25.

In some embodiments, the innate-like lymphocytes are transduced with a lentiviral vector at an MOI of about 1.

In some embodiments, the innate-like lymphocytes are transduced with a lentiviral vector at an MOI of less than 1.

Transduction efficiencies may be enhanced by using centrifugal infection (spinoculation) techniques and/or by performing transduction in the presence of transduction enhancers to increase transduction efficiency, for example, by physically reducing electrostatic repulsion between the negatively charged cell and the virion and thus increasing cell-virion interaction.

The term “transduction enhancer” may be used interchangeably with “transduction booster”.

Suitable transduction enhancers are known to those skilled in the art, and may include, VectoFusin®-1, RetroNectin®, dextran, prostaglandin E₂ (PGE₂), protamine sulphate, cyclosporine A, rapamycin, and statins (e.g. atorvastatin, fluvastatin, simvastatin, pravastatin and rosuvastatin).

The use of transduction boosters may be undesirable as they are often expensive, toxic to lymphocytes, and may hinder regulatory approval.

In some embodiments, the methods according to the invention do not involve the use of additional transduction enhancers. In other words, the innate-like lymphocytes may be incubated with lentiviral vector in an environment that is free or substantially free from transduction enhancer.

Thus, in some embodiments, the invention provides a method for introducing a nucleic acid into innate-like lymphocytes, comprising the steps of:
(i) obtaining a population of innate-like lymphocytes; and
(ii) incubating the innate-like lymphocytes with a lentiviral vector as described herein;
   or
(i) obtaining a population of innate-like lymphocytes;
(ii) activating and/or expanding said innate-like lymphocytes; and
(iii) incubating the innate-like lymphocytes with a lentiviral vector as described herein;
   wherein the lentiviral vector is incubated with the innate-like lymphocytes at an MOI of less than 5, and wherein the incubation step is free or substantially free from transduction enhancer.

The seeding density of the isolated cells during incubation with the lentiviral vector may be from about 0.01×10⁶ cells/cm² to about 1×10⁷ cells/cm², from about 0.1×10⁶ cells/cm² to about 5×10⁶ cells/cm², from about 0.25×10⁶ cells/cm² to about 5×10⁶ cells/cm², from about 0.5×10⁶ cells/cm² to about 5×10⁶ cells/cm², from about 0.5×10⁶ cells/cm² to about 4×10⁶ cells/cm², from about 0.5×10⁶ cells/cm² to about 3×10⁶ cells/cm², from about 0.5×10⁶ cells/cm² to about 2.5×10⁶ cells/cm², from about 0.5×10⁶ cells/cm² to about 2×10⁶ cells/cm², from about 0.6×10⁶ cells/cm² to about 2×10⁶ cells/cm², from about 0.7×10⁶ cells/cm² to about 2×10⁶ cells/cm², from about 0.8×10⁶ cells/cm² to about 2×10⁶ cells/cm², from about 0.9×10⁶ cells/cm² to about 2×10⁶ cells/cm², from about 1×10⁶ cells/cm² to about 2×10⁶ cells/cm², or from about 1.5×10⁶ cells/cm² to about 2×10⁶ cells/cm².

In a preferred embodiment, seeding density of the isolated cells during incubation with the lentiviral vector is 1×10⁶ cells/cm² The cells may be incubated with the lentiviral vector on, for example, day 1, 2, 3, 4, 5, 6, 7, 8, 9, or 10 of the expansion/activation step. Preferably, the cells are incubated with lentiviral vector on day 2 post-expansion.

The duration of the incubation period may be from 1 day to about 10 days, from 1 day to about 9 days, from 1 day to about 8 days, from 1 day to about 7 days, from 1 day to about 6 days, from 1 day to about 5 days, from 1 day to about 4 days, from about 2 days to about 10 days, from about 2 days to about 8 days, from about 2 days to about 7 days, about 3 days to about 7 days, or about 4 to about 6 days.

Throughout the incubation period the culture medium may be removed and/or refreshed, or supplemented with a feed medium, such as a feed medium containing cytokines and/or other suitable agents.

### Transduction Efficiency

Transduction efficiency may be determined by the expression of the gene of interest in the target cell (i.e. the innate-like lymphocyte). Expression of the gene of interest may refer to mRNA expression and/or protein expression. For example, expression of a protein encoded for by the gene of interest may be determined by FACS analysis.

Transduction efficiency may be determined by expression of a reporter gene.

Expression of the gene of interest in innate-like lymphocytes transduced according to the methods of the present invention may be about 30% to about 100%, about 40% to about 100%, or about 50% to about 100% at day 4 post-transduction.

Expression of the gene of interest in the target cell in innate-like lymphocytes transduced according to the methods present invention may be about 40% to about 100%, about 50% to about 100%, or about 60% to about 100% at day 8 post-transduction.

Expression of the gene of interest in the target cell in innate-like lymphocytes transduced according to the methods of the present invention may be about 50% to about 100%, about 60% to about 100%, or about 70% to about 100% at day 11 post-transduction.

In some embodiments, the transduction efficiencies achieved in innate-like lymphocytes transduced according to the methods of the present invention are approximately 1-, 2-, 3-, 4- or 5-fold higher than transduction efficiencies achieved with VSVg-pseudotyped lentivirus.

In some embodiments, the transduction efficiency achieved in innate-like lymphocytes transduced according to the methods of the present invention is approximately 2-fold higher than transduction efficiencies achieved with VSVg-pseudotyped lentivirus at day 7 post-transduction.

In some embodiments, the transduction efficiency achieved in innate-like lymphocytes transduced according to the methods of the present invention is approximately 4-fold higher than transduction efficiencies achieved with VSVg-pseudotyped lentivirus at day 7 post-transduction.

In some embodiments, the methods of the present invention have reduced cytotoxicity, leading to higher cell product yield and fitness.

In some embodiments, the number of viable innate-like lymphocytes at day 7 post-transduction is approximately 2-fold higher than the number achieved with with VSVg-pseudotyped lentivirus.

### Nucleic Acid/Gene of Interest

The lentiviral vector used in the methods of the invention may comprise a nucleic acid. The nucleic acid may encode one or more gene(s) of interest.

The nucleic acid may comprise DNA or RNA and may be single-stranded or double-stranded. It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the polynucleotides of the invention to reflect the codon usage of any particular host organism in which the polypeptides of the invention are to be expressed.

The polynucleotides may be modified by any method available in the art. Such modifications may be carried out in order to enhance the in vivo activity or lifespan of the polynucleotides of the invention.

The term “gene of interest” as used herein refers to a nucleotide sequence having any therapeutic or diagnostic application. The term is used interchangeably with the term “nucleotide of interest”.

The gene of interest may encode an immunologically or metabolically active protein. The protein may be naturally occurring or synthetic (i.e. engineered), for example a chimeric protein. The protein may be of human or non-human origin.

The protein may be expressed intracellularly, at the cell membrane or secreted from the cell in to which the gene is introduced.

In the present invention, innate-like lymphocytes may be transduced with a gene of interest in order to genetically modify the cells to express molecules increasing homing into tumours and/or to deliver inflammatory mediators into the tumour microenvironment, including but not limited to cytokines, soluble immune-regulatory receptors and/or ligands.

In some embodiments, the innate-like lymphocytes have been genetically modified to enhance their anti-tumour properties, such as cytotoxicity and cytokine production. For example, the gene of interest may encode a perforin, protease, toxin or cytokine.

In some embodiments, the innate-like lymphocytes have been genetically modified to enhance their proliferation and/or persistence. For example, to disrupt signalling pathways involved in exhaustion or senescence.

In some embodiments, the innate-like lymphocytes have been genetically modified to enhance their metabolic fitness. For example, the nucleic acid may encode a gene conferring increased resistance to hypoxia or glucose starvation.

In some embodiments, the innate-like lymphocytes have been genetically modified to enhance their ability to distinguish between healthy and malignant tissue. For example, the gene(s) of interest may encode a ‘NOT gate’. Innate-like lymphocytes may be activated by a first receptor binding to an antigen (e.g. an antigen expressed on malignant tissue). In ‘NOT gated’ cells, the binding of a second receptor to an alternative antigen (e.g. an antigen expressed on healthy tissue) functions to override the activating signal.

In some embodiments, the innate-like lymphocytes have been genetically modified to enhance their anti-inflammatory properties. For example, the gene of interest may encode IL-10 or TGF-beta, for secretion by the engineered cell.

Accordingly, suitable genes of interest include, but are not limited to, sequences encoding enzymes, co-factors, cytokines, chemokines, hormones, antibodies, designed ankyrin repeat proteins (DARPins), nanobodies, tribodies, duabodies, bi-specific T-cell engagers (BiTEs), engineered immunoglobulin-like molecules, single chain antibodies, fusion proteins, such as scFv-Fc fusion proteins (SFP), immune co-stimulatory molecules, immunomodulatory molecules, anti-oxidant molecules, chimeric antigen receptors, chimeric co-stimulatory receptors, dual-CARs, tri-CARs, tether-CARs, non-signalling CARs, truncated CARs, T cell receptors, a transdomain negative mutant of a target protein, toxins, conditional toxins, antigens, transcription factors, structural proteins, integrins, lectins, adhesion molecules, cell surface receptors, cell surface ligands, reporter proteins, subcellular localization signals, intracellular signalling molecules, tumour suppressor proteins, growth factors, membrane proteins, glucose transporters, ion transporters, receptors, vasoactive proteins and peptides, anti-viral proteins and ribozymes, and derivatives thereof (such as derivatives with an associated reporter group).

In some embodiments, the gene of interest may encode a therapeutic protein or combination of therapeutic proteins.

In the present invention, innate-like lymphocytes may be transduced with a gene of interest in order to genetically modify the cells to prevent or reduce expression of a target gene. For example, in order to genetically modify the cells to render them resistant to chemotherapeutic agents and/or immune-checkpoints and/or to disrupt signalling pathways.

In some embodiments, the gene of interest may encode a site-directed nuclease, for example a Cas nuclease and guide RNA (gRNA).

In some embodiments, the gene of interest (or nucleotide of interest) is a small interfering RNA (siRNA) or mircoRNA (miRNA).

### Chimeric Antigen Receptor (CAR)

CARs are proteins which, in their usual format, graft the specificity of a monoclonal antibody (mAb) to the effector function of the cell. Their usual form is that of a type I transmembrane domain protein with an antigen recognizing amino terminus, a spacer, a transmembrane domain all connected to a compound endodomain which transmits cell survival and activation signals.

The most common form of these molecules use single-chain variable fragments (scFv) derived from monoclonal antibodies to recognize a target antigen. The scFv is fused via a spacer and a transmembrane domain to a signaling endodomain. Such molecules result in activation of the cell in response to recognition by the scFv of its target. When T cells express such a CAR, they recognize and kill target cells that express the target antigen. Several CARs have been developed against tumour associated antigens, and adoptive transfer approaches using such CAR-expressing T cells are currently in clinical trial for the treatment of various cancers.

In some embodiments, the innate-like lymphocytes have been genetically modified to express a chimeric antigen receptor (CAR), chimeric co-stimulatory receptor, dual-CAR, tri-CAR, tether-CAR, non-signalling CAR, and/or truncated CAR.

### T Cell Receptor (TCR)

The TCR is the molecule found on the surface of certain lymphocytes, such as T cells, that is responsible for recognizing antigens bound to MHC molecules. The TCR heterodimer consists of either α and β chains or γ and δ chains.

Engagement of the TCR with antigen and MHC (peptide-MHC/pMHC) results in activation of its lymphocyte through a series of biochemical events mediated by associated enzymes, co-receptors, and specialized accessory molecules.

Each chain of the TCR is a member of the immunoglobulin superfamily and possesses one N-terminal immunoglobulin (Ig)-variable (V) domain, one Ig-constant (C) domain, a transmembrane domain, and a short cytoplasmic tail at the C-terminal end.

The variable domain of both the TCR a chain and p chain have three hypervariable or complementarity determining regions (CDRs). CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. Framework regions (FRs) are positioned between the CDRs. These regions provide the structure of the TCR variable region.

The repertoire of TCR variable regions is generated by combinatorial joining of variable (V), joining (J) and diversity (D) genes; and by N region diversification (nucleotides inserted by the enzyme deoxynucleotidyl-transferase).

α chains are formed from recombination events between the V and J segments. β chains are formed from recombination events involving the V, D and J segments.

The human TCRα locus, which also includes the TCRδ locus, is located on chromosome 14 (14q11.2). The TCRβ locus is located on chromosome 7 (7q34). The variable region of the TCRα chain is formed by recombination between one of 46 different Vα (variable) segments and one of 58 Ja (joining) segments (Koop et al.; 1994; Genomics; 19: 478-493). The variable region of a TCRβ chain is formed from recombination between 54 Vβ, 14 JP and 2 DP (diversity) segments (Rowen et al.; 1996; Science; 272:1755-1762).

The V and J (and D as appropriate) gene segments for each TCR chain locus have been identified and the germline sequence of each gene is known and annotated (for example see Scaviner & Lefranc; 2000; Exp Clin Immunogenet; 17:83-96 and Folch & Lefranc; 2000; Exp Clin Immunogenet; 17:42-54).

Methods for generating TCRs and affinity enhanced TCRs are known in the art. Affinity enhanced TCRs are TCRs with enhanced affinity for a peptide-MHC complex. Methods include e.g. the isolation of TCR genes that encode TCRs from patient samples (e.g. patient peripheral blood or tumour infiltrating lymphocytes), and the improvement of TCR affinity for a peptide-MHC complex via modification of TCR sequences (e.g. by in vitro mutagenesis and selection of enhanced affinity (or affinity matured) TCRs). Methods of identifying optimal-affinity TCRs involving the immunisation of antigen-negative humanised transgenic mice which have a diverse human TCR repertoire (e.g. TCR/MHC humanised mice such as ABabDII mice) with antigen, and isolation of antigen-specific TCRs from such immunised transgenic mice are also known in the art (see e.g. Obenaus M et al., Nat Biotechnol. 33(4):402-7, 2015).

In some embodiments, the gene of interest may encode a T cell receptor, such as an as T cell receptor.

### Immune Checkpoint

In some embodiments, the innate-like lymphocyte population or composition according to the invention has been genetically modified to render them resistant to immune-checkpoints.

For example, the innate-like lymphocytes may be genetically modified to prevent the expression of immune checkpoints including but not limited to PD-1, Lag-3, Tim-3, TIGIT, BTLA, CTLA-4 and combinations of these.

### Chemotherapeutic Agent

In some embodiments, the innate-like lymphocyte population or composition according to the invention has been genetically modified to increase their resistance to chemotherapeutic drugs.

A chemotherapeutic entity as used herein refers to an entity which is destructive to a cell, that is the entity reduces the viability of the cell. The chemotherapeutic entity may be a cytotoxic drug. A chemotherapeutic agent contemplated includes, without limitation, alkylating agents, anthracyclines, epothilones, nitrosoureas, ethylenimines/methylmelamine, alkyl sulfonates, alkylating agents, antimetabolites, pyrimidine analogs, epipodophylotoxins, enzymes such as L-asparaginase; biological response modifiers such as IFNα, IL-2, G-CSF and GM-CSF; platinum coordination complexes such as cisplatin, oxaliplatin and carboplatin, anthracenediones, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide.

### Composition

The present invention further provides a composition which comprises a population of innate-like lymphocytes according to the invention.

The innate-like lymphocyte composition may be a pharmaceutical composition comprising the innate-like lymphocytes as defined herein. The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

### Treatment

The term “treatment” relates to the therapeutic use of the innate-like lymphocyte population or composition according to the present invention. Herein the innate-like lymphocyte population or composition may be administered to a subject having an existing disease or condition in order to lessen, reduce or improve at least one symptom associated with the disease and/or to slow down, reduce or block the progression of the disease. The term refers to both treatment of an existing disease or prevention of a disease, i.e. prophylaxis. It will therefore be recognized that treatment, as referred to herein may in some embodiments be prophylactic.

The invention provides for a method for treating or preventing a disease in a subject, comprising administering the innate-like lymphocyte population or composition according to the invention to a subject.

The invention provides for an innate-like lymphocyte population or composition according to the invention, for use in treating or preventing a disease in a subject.

The invention provides for an innate-like lymphocyte population or composition according to the invention, for use in immunotherapy.

The invention provides for use of an innate-like lymphocyte population or composition according to the invention, for the preparation of a medicament for treatment or prevention of a disease in a subject.

In a preferred embodiment of the present invention, the subject described herein is a mammal, preferably a human, cat, dog, horse, donkey, sheep, pig, goat, cow, mouse, rat, rabbit or guinea pig, but most preferably the subject is a human.

In some embodiments, the innate-like lymphocytes are derived from the subject to be treated (autologous).

In some embodiments, the innate-like like lymphocytes are derived from a donor (allogeneic).

In one embodiment, the disease is cancer.

The cancer may be, for example, bladder cancer, gastric cancer, oesophageal cancer, breast cancer, colorectal cancer, cervical cancer, ovarian cancer, endometrial cancer, kidney cancer (renal cell), lung cancer (small cell, non-small cell and mesothelioma), brain cancer (e.g. gliomas, astrocytomas, glioblastomas), melanoma, lymphoma, small bowel cancers (duodenal and jejunal), leukemia, pancreatic cancer, hepatobiliary tumours, germ cell cancers, prostate cancer, head and neck cancers, thyroid cancer and sarcomas.

The cancer may be a paediatric cancer, for example, craniopharyngioma, ependymoma, Ewing's sarcoma, medulloblastoma, neuroblastoma, soft-tissue sarcoma or Wilms' tumour.

In some embodiments, the cancer is a paediatric cancer.

Treatment using the compositions and methods of the present invention may also encompass targeting circulating tumour cells, metastases derived from the tumour and/or liquid tumours.

Treatment with the innate-like lymphocyte population or composition of the present invention may help prevent the evolution of therapy resistant tumour cells which often occurs with standard approaches.

The methods and uses for treating cancer according to the present invention may be performed in combination with additional cancer therapies. In particular, the innate-like lymphocyte population or composition according to the present invention may be administered in combination with checkpoint blockade therapy, co-stimulatory antibodies, chemotherapy and/or radiotherapy, targeted therapy or monoclonal antibody therapy.

Checkpoint inhibitors include, but are not limited to, PD-1 inhibitors, PD-L1 inhibitors, Lag-3 inhibitors, Tim-3 inhibitors, TIGIT inhibitors, BTLA inhibitors and CTLA-4 inhibitors, for example. Co-stimulatory antibodies deliver positive signals through immune-regulatory receptors including but not limited to ICOS, CD137, CD27, OX-40 and GITR.

‘In combination’ may refer to administration of the additional therapy before, at the same time as or after administration of the innate-like lymphocyte population or composition according to the present invention.

In one embodiment, the disease is an autoimmune disease or an immune pathology.

The autoimmune disease may be for example, celiac disease, diabetes mellitus type 1, Graves' disease, inflammatory bowel disease, Crohn's disease, multiple sclerosis, psoriasis, rheumatoid arthritis, myasthenia gravis, or systemic lupus erythematosus.

In one embodiment, the disease is associated with solid organ and/or haematopoietic stem cell transplantation, for example transplant rejection or graft-versus-host disease (GvHD).

In one embodiment, the disease is an infectious disease, such as a bacterial, fungal or viral infection.

In one embodiment, the disease is a wound, ulcer, or abscess. The innate-like lymphocyte population or composition according to the invention may be used in order to aid wound healing.

The invention will now be further described by way of Examples, which are meant to serve to assist one of ordinary skill in the art in carrying out the invention and are not intended in any way to limit the scope of the invention.

### EXAMPLES

### Example 1—RD-Pro Transduces Vδ2+ Cells More Efficiently than VSVG

PBMCs were plated at 1×10⁶ cells per cm² in 96-well plates. They were stimulated with 5 μM zoledronic acid and 100 IU/ml IL-2. On day 2 cells were transduced with GFP containing RD-Pro-pseudotyped virus or VSVg-pseudotyped virus or a mock transduction (no virus) at MOI 15. On day 4, 50% of the media was removed and IL-2 was refreshed.

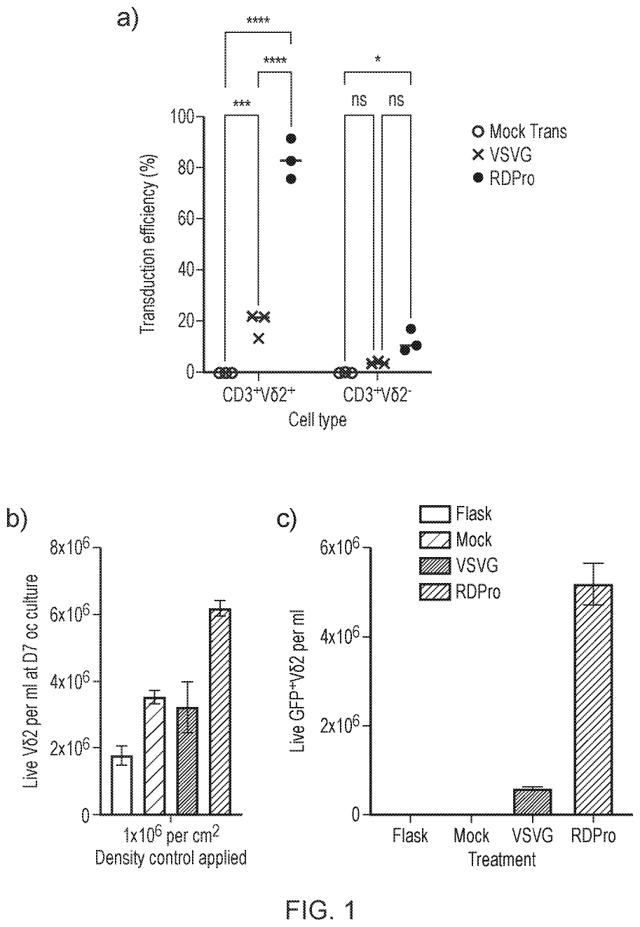

FIG. 1a demonstrates that on day 5 post-transduction (day 7 of expansion), CD3+Vδ2+ cells were transduced significantly more efficiently by RD-Pro than VSVg. There was no difference in the transduction efficiency between RD-Pro and VSVg in CD3+Vδ2− cells.

FIGS. 1b and c demonstrate that transduction with RD-Pro results in more live cells, and more GFP-expressing live cells per ml at day 7 of expansion (day 5 post-transduction) than transduction with VSVg-pseudotyped virus.

### Example 2—Efficient Transduction Achieved at Low MOI

PBMCs were plated at 1×10⁶ cells per cm² in 96-well plates and stimulated with 5 μM zoledronic acid and 100 IU/ml IL-2. On day 2 cells were transduced with RD-Pro pseudotyped bicistronic eGFP-Luciferase lentiviral vector at MOIs of 10, 5, 2.5, and 1.25.

CD3+Vδ2+ cells were analysed for GFP expression on days 4, 8 and 11 post-transduction.

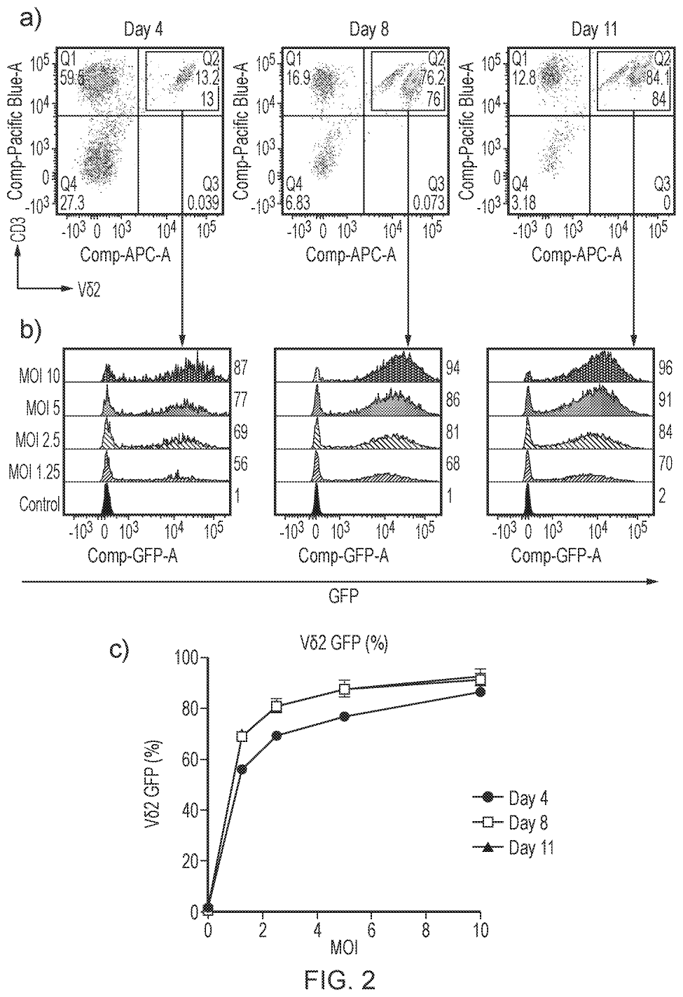

FIG. 2b demonstrates that an MOI of 1.25 generated approximately 70% transduction efficiency by days 8 and 11 post-transduction. A transduction efficiency of over 90% was achieved with MOI 10 by days 8 and 11 post-transduction.

FIG. 2c demonstrates the effect of a range of MOIs (1.25, 2.5, 5 and 10) on the transduction efficiency of Vδ2+γδ T cells at days 4, 8 and 11 following transduction. Stable transduction is achieved at day 8 post-transduction.

### Example 3—Transduction of Vδ1+, Vδ2+ and Vδ1−/Vδ2− γδ T Cells

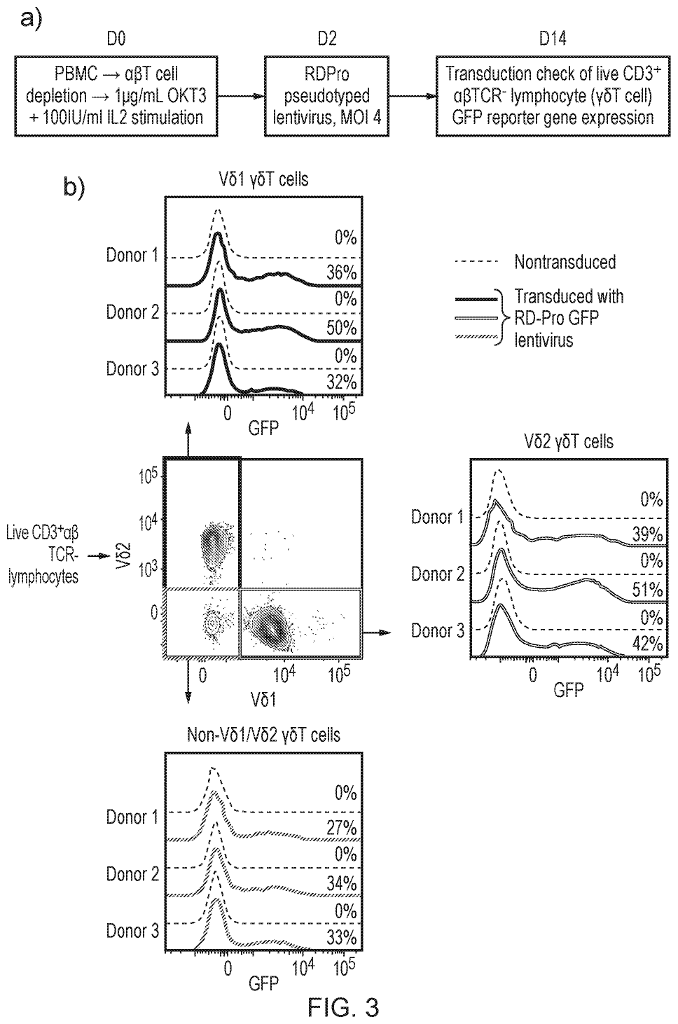

PBMCs were subjected to an as T cell depletion using magnetic-bead based separation according to the manufacturers protocol (Miltenyi). The resulting as T cell depleted PBMC were plated at 1×10⁶ cells per cm² in 96-well plates. FIG. 3a shows the method whereby the cells were stimulated with 1 μg/ml anti-CD3 antibody (OKT3) and 100 IU/ml IL-2. On day 2, cells were transduced with bicistronic GFP-luciferase containing RD-Pro-pseudotyped virus at MOI 4. Transduction efficiency of live γδ T cells (defined as CD3+/αβ TCR−) from three subsets (Vδ1+, Vδ2+ and Vδ1−/Vδ2−) was determined by flow cytometry at day 14 of expansion (day 12 post-transduction).

FIG. 3b shows the expression of GFP in Vδ1+, Vδ2+ and Vδ1−/Vδ2− γδ T cell subsets from three representative donors.

Example 4—Transduction of Vδ2+ cells with tricistronic construct PBMCs were plated at 1×10⁶ cells per cm² in 96-well plates. They were stimulated with 5 μM zoledronic acid and 100 IU/ml IL-2. On day 2, cells were transduced with GFP-Luciferase (104) or GFP-14G2a (14G2a) containing RD-Pro-pseudotyped virus, where 14G2a is a secreted opsonin targeting the tumour associated antigen GD2, at a range of MOIs (1, 2, 4, and 10 for 14G2a and 5, 10, and 20 for 104). IL-2 was refreshed every 2-3 days. At day 12 following transduction, transduction efficiency was assessed using flow cytometry and supernatant from γδ T cells transduced to express the 14G2a opsonin was collected for analysis of secreted protein content.

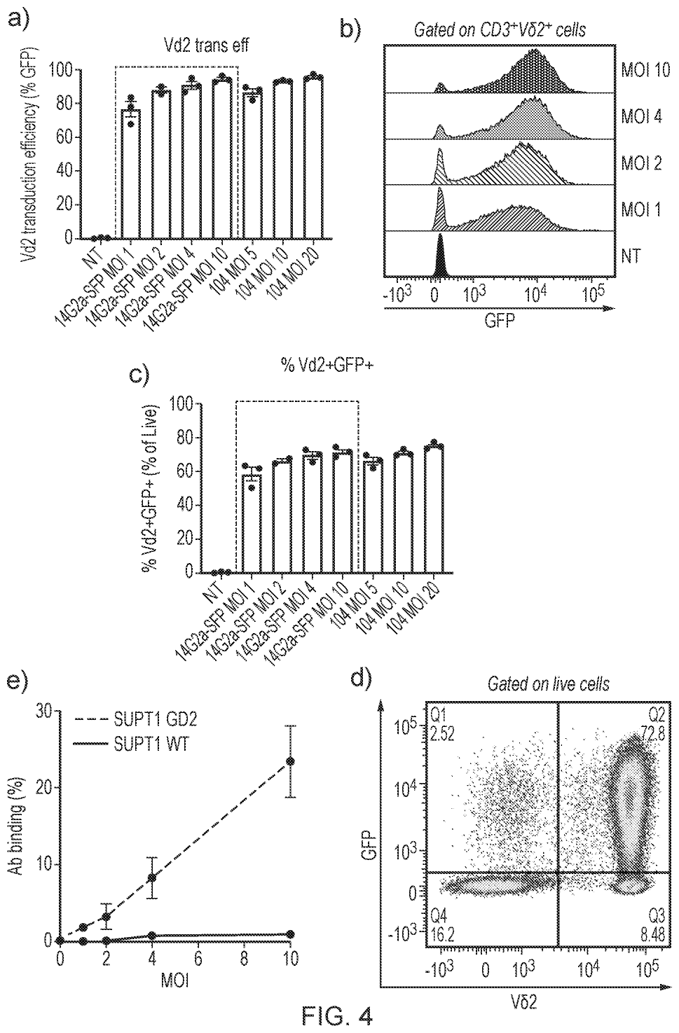

FIG. 4a shows the transduction efficiency of Vδ2+ cells transduced with either eGFP-14G2a or eGFP-Luciferase containing lentivirus.

FIG. 4b shows FACS analysis of GFP expression in γδ T cells transduced with eGFP-14G2a containing lentivirus at a range of MOIs.

FIG. 4c shows the percentage of live cells co-expressing TCR Vδ2 and GFP following transduction with either lentivirus, and FIG. 4d shows GFP and Vδ2 expression in a representative FACS plot of cells transduced with the eGFP-14G2a lentivirus.

FIG. 4e shows detection of the secreted anti-GD2 opsonin in the supernatant of cells transduced with the eGFP-14G2a lentivirus. Opsonin was detected by incubating the supernatant with either GD2+ or GD2− target cells and then detecting opsonin binding using a secondary antibody. Opsonin binding is shown to be specific, and the amount of opsonin produced is proportional to the MOI of virus used.

### Example 5—Transduction of NK Cells with Bicistronic Construct

PBMCs were plated at 1×10⁶ cells per cm² in 96-well plates. They were stimulated with 5 μM zoledronic acid and 100 IU/ml IL-2. On day 2, cells were transduced with a GFP-mitogen containing bicistronic RD-Pro-pseudotyped virus at an MOI of 2. IL-2 was refreshed every 2-3 days. At day 14 following transduction, transduction efficiency was assessed using flow cytometry. NK cells were counted as CD3-negative and CD56-positive cells in culture.

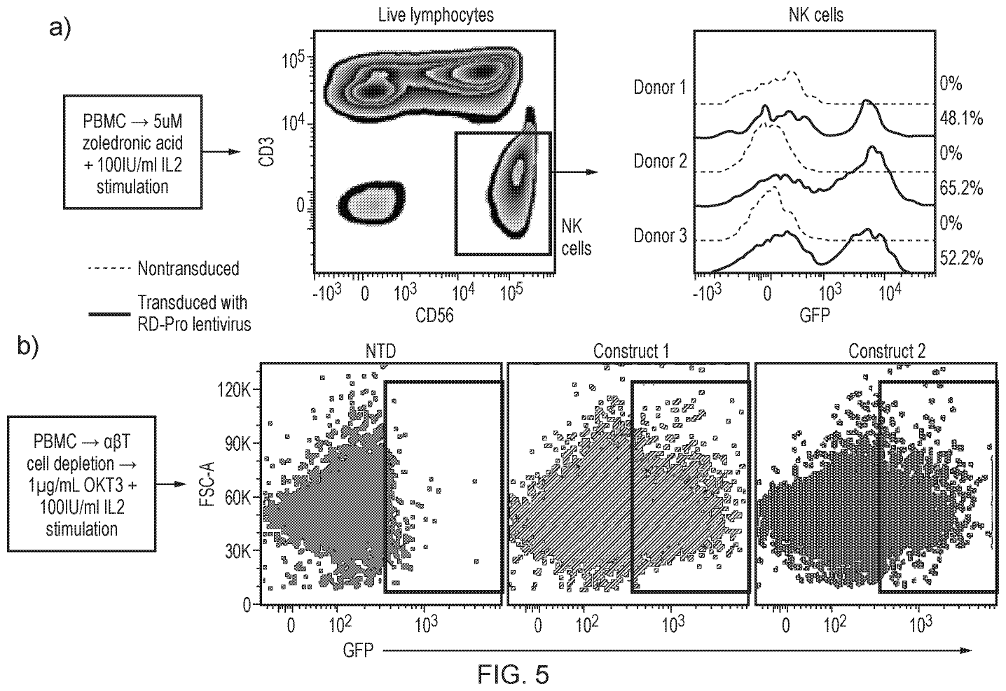

FIG. 5a shows the transduction efficiency of NK cells from 3 independent donor expansions, achieving a mean transduction efficiency of ˜55%.

PBMCs were subjected to an as T cell depletion using magnetic-bead based separation according to the manufacturers protocol (Miltenyi). The resulting as T cell depleted PBMC were plated at 1×10⁶ cells per cm² in 96-well plates. FIG. 3a shows the method whereby the cells were stimulated with 1 μg/ml anti-CD3 antibody (OKT3) and 100 IU/ml IL-2. On day 2, cells were transduced with bicistronic GFP-luciferase containing RD-Pro-pseudotyped virus at MOI 4. Transduction efficiency of live NK cells (defined as CD3−/CD56+) from three subsets was determined by flow cytometry at day 14 of expansion (day 12 post-transduction).

FIG. 5b shows the comparative GFP expression of one representative donor NK cells either non-transduced or transduced with two different bicistronic GFP-mitogen constructs.

### Example 6—Transduction of γδ T Cells Across Different Expansion Conditions

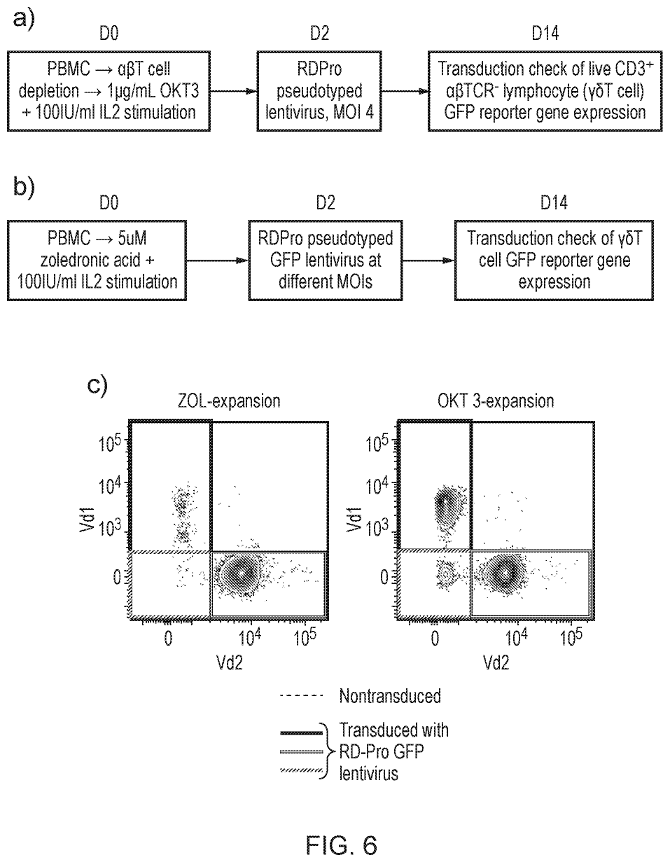

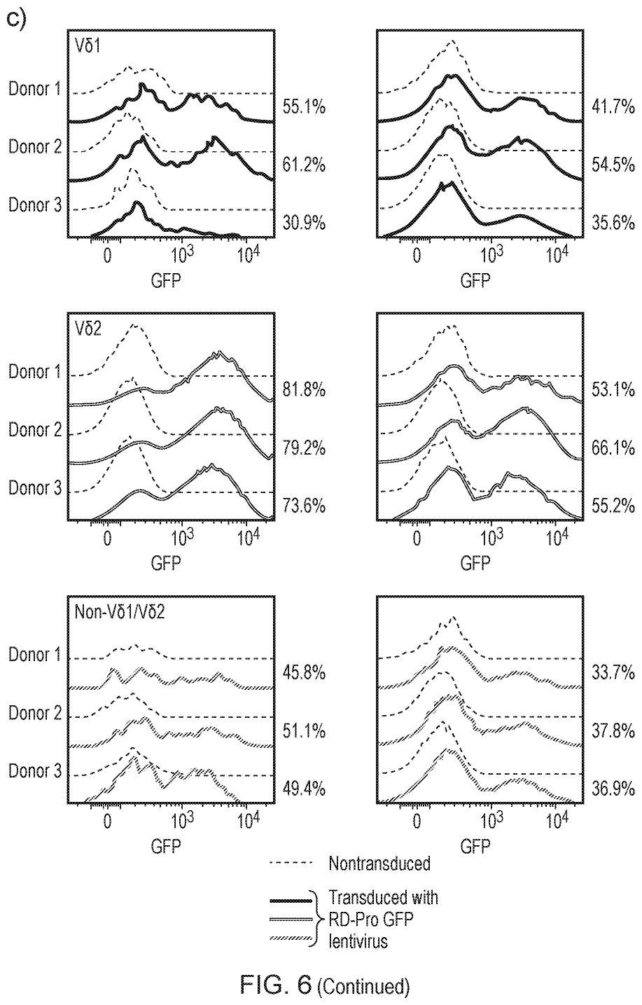

FIG. 6a shows the method described in Example 3, wherein αβ-depleted PBMCs were plated at 1×10⁶ cells per cm² in 96-well plates and stimulated with 1 μg/ml anti-CD3 antibody (OKT3) and 100 IU/ml IL-2 (OKT-3-expansion).

FIG. 6b shows a method wherein non-αβ-depleted PBMCs were plated at 1×10⁶ cells per cm² in 96-well plates and stimulated with 5 μM zoledronic acid and 100 IU/ml IL-2 (ZOL-expansion).

γδ T cells were activated using two translationally-relevant GMP-compatible protocols, and transduced with a bicistronic RD-Pro vector at MOI 4. GFP transgene expression was measured in Vδ1+, Vδ2+ and Vδ1−/Vδ2− γδ T cell subsets from three independent donors (FIG. 6c). As can be seen, RDPro effectively transduces different types of γδ T cells across different expansion protocols.

### Example 7—Efficient Transduction of NK Cells at Low MOI

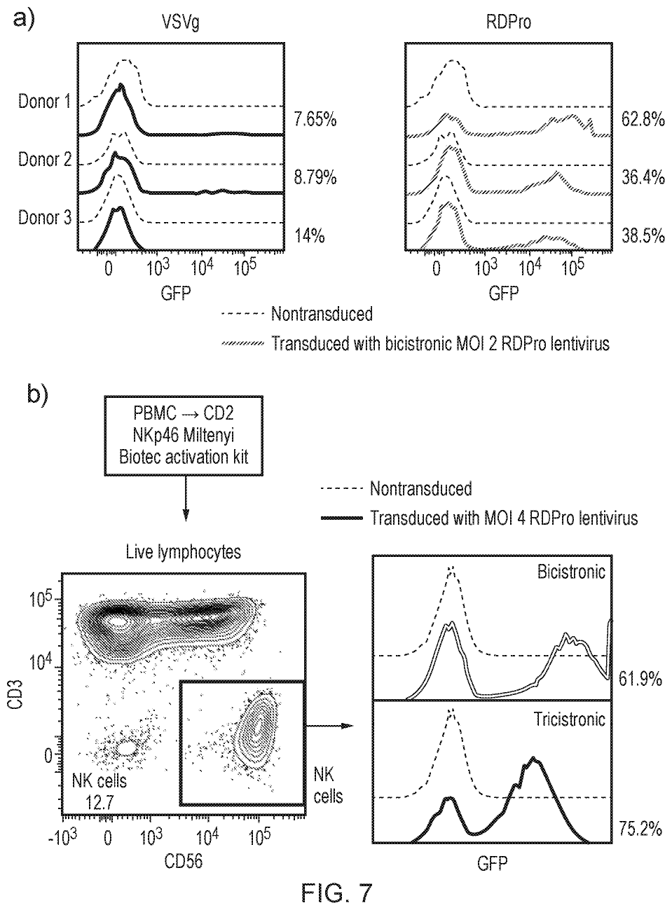

Zoledronic acid-activated PBMC were transduced at MOI 2 with RD-Pro- and VSVg-pseudotyped lentivirus comprising a bicistronic construct. NK cells were highly GFP positive at harvest of the culture at day 14 when RD-Pro lentivirus was used (mean transduction efficiency of ˜46%), but not when VSVg-pseudotyped lentivirus was used (mean transduction efficiency of ˜10%) (FIG. 7a).

High transduction was maintained with large bicistronic and immunologically active tricistronic vectors at low MOI when NK cells were activated with proprietary, feeder-free and CD2/NKp46 GMP-compatible NK cell manufacturing kits from Miltenyi Biotec.

### Example 8—RD-Pro Pseudotyped Lentivirus Transduces NK-T Cells to Translationally Relevant Efficiency at Low MOIs

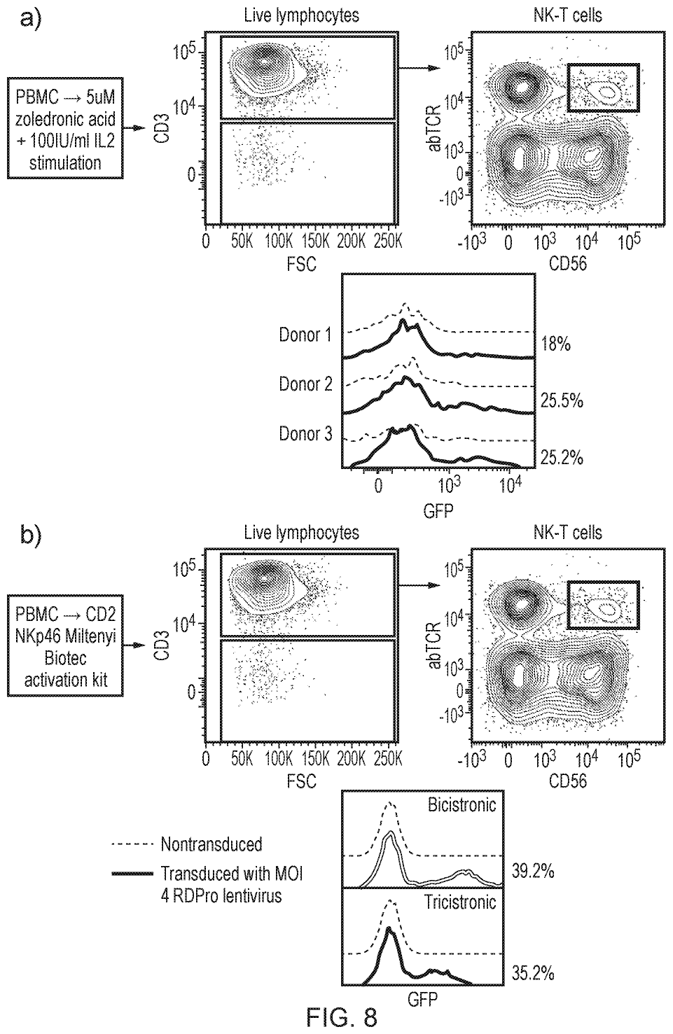

Zoledronic acid-activated PBMC were transduced at MOI 4 with RD-Pro-pseudotyped lentivirus. NK T-cells (CD3+CD56+αβTCR+) were GFP positive at harvest of the culture at day 14 (FIG. 8a).

High NK-T cell transduction was maintained with large bicistronic and immunologically active tricistronic vectors at low MOI when NK cells were activated with proprietary, feeder-free and CD2/NKp46 GMP-compatible NK cell manufacturing kits from Miltenyi Biotec (FIG. 8b).

1. A method for introducing nucleic acid into innate-like lymphocytes with a lentiviral vector, wherein the lentiviral vector comprises a RD114-HIV chimeric envelope protein.

2. The method according to claim 1, wherein the innate-like lymphocytes are γδ T cells, Vδ1+γδ T cells, Vδ2+γδ T cells, non-Vδ1+/Vδ2+γδ T cells, NK cells or NKT cells, natural killer (NK) cells, natural killer T (NKT) cells, invariant natural killer T (iNKT) cells, mucosal-associated invariant T (MAIT) cells, lymphoid tissue inducer (LTi) cells, intra-epithelial lymphocytes (IELs), innate lymphoid cells (ILC), including group 1 innate lymphoid cells, group 2 innate lymphoid cells, group 3 innate lymphoid cells, B1 cells, marginal zone B cells, and/or cells expressing CD8αα, or the innate-like lymphocytes are not canonical as T cells.

3-9. (canceled)

10. The method according to claim 1, wherein the chimeric envelope protein comprises a RD114 envelope protein, wherein the R peptide cleavage sequence is replaced with a HIV-1 matrix/capsid cleavage sequence.

11. The method according to claim 1, wherein the chimeric envelope protein comprises a sequence having at least 60%, at least 70%, at least 80%, at least 90% identity to SEQ ID NO: 4.

12. The method according to claim 1, wherein the nucleic acid is introduced in to the innate-like lymphocytes by said lentiviral vector at a multiplicity of infection (MOI) of around 50, around 25, around 10, around 5, around 2.5 or around 1.25.

13. The method according to claim 1, wherein the nucleic acid encodes a gene of interest.

14. The method according to claim 13, wherein the gene encodes an immunologically or metabolically active naturally occurring or synthetic protein or chimeric molecule of human or non-human origin, expressed either intracellularly, at the cell membrane or secreted from the cell.

15. The method according to claim 13, wherein the gene encodes a chimeric antigen receptor (CAR), chimeric co-stimulatory receptor, dual-CAR, tri-CAR, tether-CAR, non-signalling CAR, truncated CAR, T cell receptor (TCR), TCR co-expressed with CAR, scFv-Fc fusion protein (SFP), antibody, DARPIN, nanobody, tribody, duabody, bi-specific T-cell engager (BiTE), transcription factor, intracellular signalling molecule and/or mediator, cytokine, chemokine, integrin, lectin, adhesion molecule, cell surface receptor, cell surface ligand, glucose transporter, ion transporter, membrane-proximal and/or membrane-distal intracellular enzyme, such as a phosphatase, tyrosine kinase, serine threonine kinase, protease, matrix metalloproteinase, and/or degron.

16. A method for introducing nucleic acid into innate-like lymphocytes according to claim 1, wherein the method comprises the steps of:
obtaining a population of innate-like lymphocytes; and
incubating the innate-like lymphocytes with a lentiviral vector;
wherein the lentiviral vector comprises a RD114-HIV chimeric envelope protein.

17. The method according to claim 16, wherein the incubation step is carried out in the absence of transduction enhancer(s).

18. The method according to claim 16, wherein the population of innate-like lymphocytes are isolated from a blood or tissue-derived human product.

19. The method according to claim 16, wherein the population of innate-like lymphocytes are stimulated, such as activated and/or expanded, prior to and/or during incubation with said lentiviral vector.

20. (canceled)

21. A population of innate-like lymphocytes, wherein said innate-like lymphocytes have been genetically engineered with a lentiviral vector comprising a RD114-HIV chimeric envelope protein.

22. A composition comprising an innate-like lymphocyte population according to claim 21.

23-24. (canceled)

25. An innate-like lymphocyte population according to claim 21, wherein the innate-like lymphocytes are at least 30%, at least 40%, at least 50%, at least 60%, at least 70%, at least 80%, or at least 90%: γδ T cells, Vδ1+γδ T cells, Vδ2+v T cells, non-Vδ1+/Vδ2+v T cells, NK cells or NKT cells.

26-30. (canceled)

31. An innate-like lymphocyte population or composition according to claim 21, wherein the innate-like lymphocytes have been genetically modified to: enhance their cytotoxicity and cytokine production; to enhance their proliferation and/or persistence; to enhance their resistance to hypoxia or glucose starvation; to enhance their ability to distinguish between healthy and malignant tissue; to enhance their anti-inflammatory properties via the secretion of IL-10 or TGF-beta; or to increase their resistance to chemotherapeutic drugs.

32-36. (canceled)

37. An innate-like lymphocyte population or composition according to claim 21, wherein the innate-like lymphocytes have been genetically modified to express a chimeric antigen receptor (CAR), chimeric co-stimulatory receptor, dual-CAR, tri-CAR, tether-CAR, non-signalling CAR, truncated CAR, T cell receptor (TCR), TCR co-expressed with CAR, scFv-Fc fusion protein (SFP), antibody, DARPIN, nanobody, tribody, duabody, bi-specific T-cell engager (BiTE), transcription factor, intracellular signalling molecule and/or mediator, cytokine, chemokine, integrin, lectin, adhesion molecule, cell surface receptor, cell surface ligand, glucose transporter, ion transporter, membrane-proximal and/or membrane-distal intracellular enzyme, such as a phosphatase, tyrosine kinase, serine threonine kinase, protease, matrix metalloproteinase, and/or degron.

38. A method of treating or preventing a disease in a subject, comprising administering the innate-like lymphocyte population or composition according to claim 21 to a subject.

39. The method according to claim 38, wherein the innate-like lymphocytes are allogeneic.

40-44. (canceled)

45. The method according to claim 38, wherein the disease is cancer, an autoimmune disease or immune pathology, an infectious disease, or a disease is associated with organ transplant, or the disease is a wound, ulcer, or abscess.

46-49. (canceled)


SEQUENCE LISTING (4 sequences)

SEQ ID NO: 1 - protein, 564 aa, synthetic construct
  source 1..564: note = RD114 envelope protein
MKLPTGMVILCSLIIVRAGFDDPRKAIALVQKQHGKPCECSGGQVSEAPPNSIQQVTCPGKTAYLMTNQKWKCRVTPKISPSGGELQNCPCNTFQDSMHSSCYTEYRQCRRINKTYYTATLLKIRSGSLNEVQILQNPNQLLQSPCRGSINQPVCWSATAPIHISDGGGPLDTKRVWTVQKRLEQIHKAMTPELQYHPLALPKVRDDLSLDARTFDILNTTFRLLQMSNFSLAQDCWLCLKLGTPTPLAIPTPSLTYSLADSLANASCQIIPPLLVQPMQFSNSSCLSSPFINDTEQIDLGAVTFTNCTSVANVSSPLCALNGSVFLCGNNMAYTYLPQNWTRLCVQASLLPDIDINPGDEPVPIPAIDHYIHRPKRAVQFIPLLAGLGITAAFTTGATGLGVSVTQYTKLSHQLISDVQVLSGTIQDLQDQVDSLAEVVLQNRRGLDLLTAEQGGICLALQEKCCFYANKSGIVRNKIRTLQEELQKRRESLATNPLWTGLQGFLPYLLPLLGPLLTLLLILTIGPCVFSRLMAFINDRLNVVHAMVLAQQYQALKAEEEAQD

SEQ ID NO: 2 - protein, 8 aa, synthetic construct
  source 1..8: note = R peptide
VHAMVLAQ

SEQ ID NO: 3 - protein, 8 aa, synthetic construct
  source 1..8: note = matrix/capsid cleavage peptide
SQNYPIVQ

SEQ ID NO: 4 - protein, 565 aa, synthetic construct
  source 1..565: note = RD-Pro envelope protein
MKLPTGMVILCSLIIVRAGFDDPRKAIALVQKQHGKPCECSGGQVSEAPPNSIQQVTCPGKTAYLMTNQKWKCRVTPKNLTPSGGELQNCPCNTFQDSMHSSCYTEYRQCRANNKTYYTATLLKIRSGSLNEVQILQNPNQLLQSPCRGSINQPVCWSATAPIHISDGGGPLDTKRVWTVQKRLEQIHKAMHPELQYHPLALPKVRDDLSLDARTFDILNTTFRLLQMSNFSLAQDCWLCLKLGTPTPLAIPTPSLTYSLADSLANASCQIIPPLLVQPMQFSNSSCLSSPFINDTEQIDLGAVTFTNCTSVANVSSPLCALNGSVFLCGNNMAYTYLPQNWTGLCVQASLLPDIDIIPGDEPVPIPAIDHYIHRPKRAVQFIPLLAGLGITAAFTTGATGLGVSVTQYTKLSHQLISDVQVLSGTIQDLQDQVDSLAEVVLQNRRGLDLLTAEQGGICLALQEKCCFYANKSGIVRNKIRTLQEELQKRRESLASNPLWTGLQGFLPYLLPLLGPLLTLLLILTIGPCVFSRLMAFINDRLNVSQNYPIVQQYQALKAEEEAQD